(19) United States
(12) Patent Application Publication
(10) Pub. No.: US 2026/0102368 A1
(43) Pub. Date: Apr. 16, 2026

(54) NOVEL FEED ADDITIVES COMPRISING FATTY ACID ESTERS AND RELATED METHODS TO IMPROVE ANIMAL GROWTH PERFORMANCE AND HEALTH

(71) Applicant: KEMIN INDUSTRIES, INC., DES MOINES, IA (US)

(72) Inventors: Jatuporn SALAKLANG, Clive, IA (US); Jon Kieran Rubach, West Des Moines, IA (US); Haridasan CHIRAKKAL, Herentals (BE)

(73) Assignee: KEMIN INDUSTRIES, INC., DES MOINES, IA (US)

(21) Appl. No.: 19/283,092

(22) Filed: Jul. 28, 2025

Related U.S. Application Data
(60) Provisional application No. 63/676,467, filed on Jul. 29, 2024.
(60) Provisional application No. 63/790,394, filed on Apr. 17, 2025.

(51) Int. Cl. A61K 31/225; A23K 20/105; A23K 50/10; A23K 50/20; A23K 50/30; A23K 50/40; A23K 50/75; A23K 50/80; A61K 9/00; A61P 1/00; A61P 31/04

(52) U.S. Cl. CPC A61K 31/225; A23K 20/105; A23K 50/10; A23K 50/20; A23K 50/30; A23K 50/40; A23K 50/75; A23K 50/80; A61K 9/0053; A61P 1/00; A61P 31/04

(57) ABSTRACT

The present invention relates to novel compositions, such as feed additives, for improved performance and/or intestinal health of animals and associated methods of use, where the composition has an improved odor profile compared to butyric acid alone. Another aspect of the present invention relates to novel composition of propylene glycol esters alone or applied on a carrier, which are suitable as a feed additive or dietary supplement.

### CROSS-REFERENCE TO RELATED APPLICATIONS

The present application claims the benefit of priority to U.S. Patent Application No. 63/676,467, filed Jul. 29, 2024, entitled “NOVEL FEED ADDITIVES COMPRISING FATTY ACID ESTERS AND RELATED METHODS TO IMPROVE ANIMAL GROWTH PERFORMANCE AND HEALTH,” and U.S. Patent Application No. 63/790,394, filed Apr. 17, 2025, entitled “NOVEL FEED ADDITIVES COMPRISING FATTY ACID ESTERS AND RELATED METHODS TO IMPROVE ANIMAL GROWTH PERFORMANCE AND HEALTH,” the entire disclosures of which are incorporated herein by reference in their entirety.

### BACKGROUND OF THE INVENTION

The optimal productivity and feed efficiency of animals relies on efficient nutrient uptake by the gastrointestinal tract. Irritation of the intestinal epithelia can result from diet, pathogenic infections and/or stress. This can damage the intestinal barrier resulting in increased permeability, malabsorption of nutrients, increased incidence of diarrhea, inflammation, and oxidative stress which all effect the growth and health of the animal. Increased intestinal barrier permeability results in a redistribution of energy towards suppressing the intestinal challenge and repairing the gut, which in turn reduces animal growth.

Necrotic Enteritis and Coccidiosis are major health issues related to reduced weight gain and decreased performance in broilers raised under antibiotic-free (ABF) conditions. Infection of broilers with Eimeria, specifically E. maxima, results in opportunistic growth of Clostridium perfringens in the gut, resulting in the development of Necrotic Enteritis.

The use of butyric acid, valeric acid, or the salts of these acids or other short chain fatty acids to improve the absorption of nutrients from the intestine is hindered by the very unpleasant odor of these compounds. Therefore, an effective means of masking the odor is needed in the art.

### BRIEF SUMMARY OF THE INVENTION

The present invention provides methods and compositions for using esters of short-chain fatty acids to improve the growth, intestinal health and well-being of animals, including but not limited to pets and livestock, through improving intestinal structure performance and function, which can be quantified by improved weight gain and enhanced feed efficiency. Specifically, the present invention relates to the compositions of propylene glycol esters of butyric and/or valeric acid, which improve the intestinal health of animals.

One aspect of the present invention relates to novel compositions for improved performance and/or intestinal health of animals where the composition has an improved odor profile compared to butyric acid alone. Another aspect of the present invention relates to novel composition of propylene glycol esters alone or applied on a carrier which are suitable as a feed additive or supplement.

Another aspect of the present invention relates to methods of administering the compositions to animals in an amount effective to improve the growth performance of animals. According to at least one embodiment, the composition is administered to an animal in feed or food in an amount effective to improve the intestinal health of the animals.

Another aspect of the invention relates to methods for reducing mortality due to necrotic enteritis. According to at least one embodiment, a composition of propylene glycol-butyric acid esters is administered to reduce mortality due to necrotic enteritis.

### BRIEF DESCRIPTION OF THE FIGURES

FIG. 1 is the chemical structure of tributyrin, glycerol with three butyric acid ester bonds.

FIG. 2 is the Fischer Esterification reaction scheme for glycerol and butyric acid. The acid can react with the hydroxyl groups on glycerol, creating a mixture of products. Only one of the possible reaction products is shown.

FIG. 3 is the structures of glycerol and propylene glycol.

FIG. 4 shows the possible products from the reaction of propylene glycol and butyric acid.

FIG. 5A is an HPLC chromatogram for the propylene glycol butyrate final product mixture comprising butyric acid (at 2.943 min), butyrate monoesters (at 3.157 min and 5.301 min) and butyrate diester (at 6.032 min).

FIG. 5B is the analysis of the area under the curves of the HPLC chromatogram for the propylene glycol butyrate final product mixture in FIG. 5A comprising butyric acid (at 2.943 min), butyrate monoesters (at 3.157 min and 5.301 min) and butyrate diester (at 6.032 min).

FIG. 6A is an HPLC chromatogram for the propylene glycol valerate final product mixture comprising valeric acid (at 3.158 min), valeric acid monoesters (at 3.468 min and 6.527 min) and valeric acid diester (at 9.534 min).

FIG. 6B is the analysis of the area under the curves of the HPLC chromatogram for the propylene glycol valerate final product mixture in FIG. 6A comprising valeric acid (at 3.158 min), valeric acid monoesters (at 3.468 min and 6.527 min) and valeric acid diester (at 9.534 min).

FIG. 7 is the duodenum histology analysis of villus height. Values with different letters are significantly different (P<0.05).

FIG. 8 is the duodenum histology analysis of crypt depth. Values with different letters are significantly different (P<0.05).

FIG. 9 is the duodenum histology villi height/crypt depth ratio. Values with different letters are significantly different (P<0.05).

FIG. 10 shows treatment performance with pigs from 1-42 days (Body Weight).

FIG. 11 shows treatment performance with pigs from 1-42 days (Feed Intake).

FIG. 12 shows treatment performance with pigs from 1-42 days (Feed Conversion Ratio (FCR)).

FIG. 13 shows treatment performance with pigs from 1-42 days (Feed Cost per Gain (FCG)).

FIG. 14 shows cell confluency observed with an inverted microscope at 0 hour (a-c), after 4 hours with no treatment (d-e), and after 16 hours with no treatment (f-g).

FIG. 15 shows cell confluency observed with an inverted microscope at 0 hour (a-c) and after 4 hours with 0.1 mM of valeric acid (d-f).

FIG. 16 shows cell confluency observed with an inverted microscope at 0 hour (a-c) and after 4 hours with 1.0 mM of valeric acid (d-f).

FIG. 17 shows cell confluency observed with an inverted microscope at 0 hour (a-c) and after 4 hours with 0.1 mM of monopropylene glycol valerate ester, MPG-C5 (d-f).

FIG. 18 shows cell confluency observed with an inverted microscope at 0 hour (a-c) and after 4 hours with 1.0 mM of monopropylene glycol valerate, MPG-C5 (d-f).

FIG. 19 shows cell confluency observed with an inverted microscope at 0 hour (a-c) and after 5 hours without any treatment (i.e., control) (d-f).

FIG. 20 shows cell confluency observed with an inverted microscope at 0 hour (a-c) and after 5 hours with 0.1 mM valeric acid (d-f).

FIG. 21 shows cell confluency was observed with an inverted microscope at 0 hour (a-c) and after 5 hours with 1.0 mM valeric acid (d-f).

FIG. 22 shows cell confluency observed with an inverted microscope at 0 hour (a-c) and after 5 hours with 0.1 mM MPG-C5 (d-f).

FIG. 23 shows cell confluency observed with an inverted microscope at 0 hour (a-c) and after 5 hours with 1.0 mM MPG-C5 (d-f).

FIG. 24 shows the percent compositions of butyric acid, monoester, and diester products after hydrolysis of MPG butyrate by pancreatic lipase over 120 minutes. The compositions of the hydrolysis products were measured by HPLC.

FIG. 25 shows the percent compositions of butyric acid, mono-, di-, and tri-ester products after hydrolysis of tributyrin by pancreatic lipase over 120 minutes. The compositions of the product after lipase hydrolysis were measured by HPLC.

FIG. 26 shows that MPG butyrate ester demonstrates a faster antimicrobial/E. Coli inhibition effect than tributyrin after hydrolysis by pancreatic lipase.

FIG. 27 shows an E. Coli growth curve for time point samples from the lipase hydrolysis of MPG-butyrate.

FIG. 28 shows an E. Coli growth curve for time point samples from the lipase hydrolysis of tributyrin.

### DETAILED DESCRIPTION OF THE INVENTION

The present invention relates generally to using fatty acid esters to improve the growth, intestinal health and overall wellbeing of humans, pets, and livestock. More specifically, the present invention relates to the use of propylene glycol esters of butyric or other short chain fatty acids to improve the growth performance and/or intestinal health of animals, including humans and fish, dogs, poultry, pigs, cattle, sheep, goats and horses. Such improvement may be measured, for instance, by a reduction in the adverse effects of intestinal diseases as compared with those animals not being fed such compositions.

In particular, the intestinal barrier functions in pigs are highly vulnerable to weaning stress and contamination. It was previously reported that the ingestion of mycotoxin-contaminated feed and Escherichia coli infection can lead to defective intestinal barrier function, increasing intestinal permeability and affecting nutrient absorption. This, in turn, can affect the animals' growth and performance. On the other hand, early-weaning piglets with an immature digestive system may also suffer from scratch damage to their intestinal barrier when the diet is changed to a solid diet intake.

Short-chain fatty acids such as butyric acid and valeric acid have been reported to be effective in the maintenance of gut integrity and health. However, their effects may vary due to their instability to gut enzymes. Although recent research has shown that this issue may be circumvented with esterification, the commonly used glycerol backbone can potentially be degraded into acrolein, leading to detrimental effects on animal health.

Short-chain fatty acids such as butyric and valeric acids have been reported to be effective against pathogens and may improve gut health. However, their performances may vary due to their poor stability, low resistance to gut enzymes, and absorption of the acids in the digestive tract before they reach the intestine. The direct use of these acids may also pose feed palatability and handling issues due to their pungency. Methods to address the unpleasant odor of butyric acid and other short-chain fatty acids (C2-C6) are, for example, encapsulation of butyric acid and/or the form of an ester with alcohols or carbohydrates. With this, there is an increasing focus on research regarding their esters, where short-chain organic acids are esterified, commonly with glycerol. Reports have shown that these esters could improve the intestinal barrier integrity and morphology. On the other hand, they also enable sustained release of the corresponding organic acids throughout the gastrointestinal tract.

Although glycerides can confer additional benefits compared to their organic acids, a report has shown that gut microbial glycerol metabolism may lead to acrolein production. Acrolein is highly reactive and may induce oxidative stress and immune dysfunction. In contrast, monopropylene glycol (MPG) is degraded into non-toxic by-products such as carbon dioxide and water.

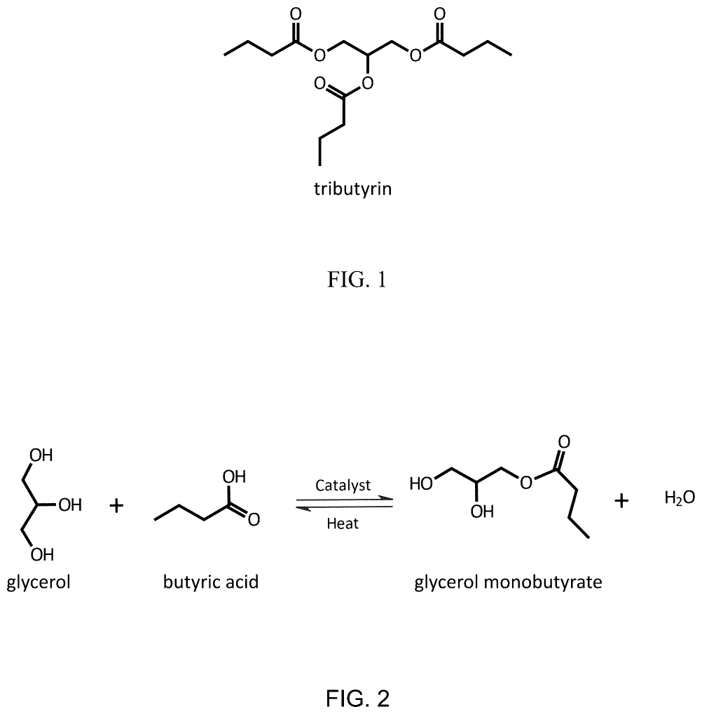

Esters of short-chain fatty acids are common in flavorings and fragrances, and the glycerol esters of long-chain fatty acids are important to the metabolism of all organisms. Triglycerides of long-chained fatty acids are energy storage molecules for all types of organisms, and tributyrin, which is a triglyceride of butyric acid is found in dairy products, such as butter (FIG. 1).

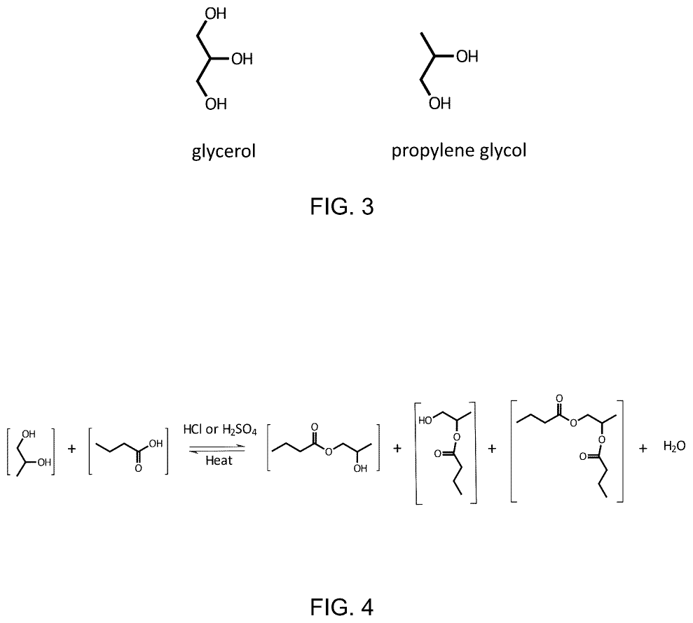

Forming esters of glycerol through esterification and transesterification is a common chemical process. The esterification of glycerol is commonly done through a Fischer esterification reaction (FIG. 2). This reaction can be used to form esters from many types of organic acids and alcohols. One key feature to this reaction is that water is the by-product. The reactants and products of this reaction are in equilibrium and the accumulation of water from the reaction can inhibit the production of monoglyceride. When glycerol is used, the large difference in viscosity and physical properties of the butyric acid and glycerol lead to non-homogeneous mixing and a lower reaction yield, as compared to other alcohols, for example, diols. Propylene glycol lacks one hydroxyl group as compared to glycerol (FIG. 3). The physical properties of propylene glycol allow for the esterification to proceed with better reaction yields, as compared to glycerol. The controlled esterification reaction of propylene glycol can lead to the formation of mono and/or diester products (FIG. 4). In general, the metabolism of short chain fatty acid esters will occur through the hydrolysis of the esters by lipase in the intestine of the animals. The hydrolysis is thought to occur on a similar timescale for various glycerol esters.

The present invention relates to a feed additive which contains a propylene glycol-butyric acid ester to improve the growth performance or health of a human or an animal. The animal may include fish, dogs, poultry pigs, cattle, sheep, goats, and horses. The composition of this feed additive has a low odor profile when compared to butyric acid alone. In various embodiments, the ester may be propylene glycol butyrate ester, propylene glycol valerate ester, or propylene glycol ester with one valeric acid and one butyric acid. In various embodiments, the ester is propylene glycol dibutyrate ester, propylene glycol monobutyrate ester, or mixtures thereof.

In certain embodiments, the esters may be delivered via feed. In an embodiment, the ester is delivered in a fat source. In an embodiment, the ester is delivered on a dry carrier. In an embodiment the ester is sprayed directly onto feed before a pelleting process. In an embodiment, the ester is sprayed directly onto the feed after pelleting.

In certain embodiments, the feed additive may contain additional components, including C6-C12 fatty acid monopropylene glycol-esters.

The present invention also relates to method of improving the growth performance or intestinal health of an animal or human. This is achieved by administering an effective amount of propylene glycol-butyric acid esters that is effective to improve the growth performance or intestinal health of the animal or human. The animal to which the ester is administered may include fish, dogs, poultry, pigs, cattle, sheep goats, and horses.

An improvement in growth performance may be evaluated by any common metric, including feed conversion ratio (FCR). In an embodiment, the FCR may be decreased by 1-20%, 1-10%, 1-5%, 1.5-2.5%, or 5-10%.

An improvement in intestinal health may be evaluated by any common metric, including villus height or crypt depth, or the ratio of the two. In an embodiment, the villus height may increase by about 1-5%, or about 2-4%. In an embodiment, the crypt depth may decrease by up to 25%, up to 15%, up to 10%, up to 5% or up to 3%. In an embodiment, the ratio of villus height to crypt depth may increase by up to 50%, up to 40%, up to 30%, up to 20%, up to 10% or up to 5%. In an embodiment, the lesion scores may be between about 0 and 1.5, about 0.5 and 1.0, or about 0.6 and 0.9.

The ester used to improve the growth performance or intestinal health of the animal or human may be propylene glycol butyrate ester, propylene glycol valerate ester or propylene glycol ester with one valeric acid and one butyric acid. In certain embodiments, the ester may be propylene glycol dibutyrate ester, propylene glycol monobutyrate ester, or mixtures thereof. The ester may be propylene glycol divalerate ester, propylene glycol monovalerate ester, or mixtures thereof.

The present invention relates to a feed additive which has an efficacious amount of low odor propylene glycol esters that have a reduced odor profile when compared to butyric acid alone.

Another aspect of the present invention relates to a feed additive which has an efficacious amount of low odor propylene glycol esters that have a lower carbon footprint than commercially available alternatives.

Another aspect of the present invention relates to a method of providing a sustainable feed additive comprising administering an efficacious amount of low odor propylene glycol ester that is more cost effective than encapsulated calcium butyrate. The ester may be propylene glycol butyrate ester, propylene glycol valerate ester or propylene glycol ester with one valeric acid and one butyric acid. The ester may be propylene glycol dibutyrate ester, propylene glycol monobutyrate ester, or mixtures thereof. The ester may be propylene glycol divalerate ester and/or propylene glycol monovalerate ester, or mixtures thereof.

In certain embodiments, the sustainable feed additive may be delivered to animals such as fish, dogs, poultry, pigs, cattle, sheep, goats, and horses. In an embodiment, the sustainable feed additive is delivered in a fat source. In an embodiment, the feed additive is delivered on a dry carrier. In an embodiment the sustainable feed additive is sprayed directly onto feed before a pelleting process. In an embodiment, the sustainable feed additive is sprayed directly onto the feed after pelleting.

In certain embodiments, the sustainable feed additive may contain additional components, including C6-C12 fatty acid monopropylene glycol-esters.

Production of propylene glycol ester requires less energy and improves the growth of animals similar to that of glycerol esters, therefore the product has a lower carbon footprint and is more sustainable compared to the production of tributyrin, encapsulated calcium butyrate (referred to herein as “Ca butyrate”) and other commercially available products.

The present invention relates to a method for repairing an intestinal barrier of a human or animal, wherein an amount of propylene glycol-butyric acid esters effective to facilitate repair of the intestinal barrier are administered to the human or animal.

The present invention relates to a method of reducing mortality due to necrotic enteritis, wherein an amount of propylene glycol-butyric acid esters effective to reduce the mortality due to necrotic enteritis is administered. In an embodiment, the percentage of necrotic enteritis mortality may be reduced to between 0 and 15%, 1 and 5%, 5.0 and 10%, or 5.0 and 7.5%.

### EXAMPLES

### Example 1—Improved Growth Performance of Chickens Fed the Propylene Glycol Esters

A 42-day trial with 2,400 Cobb×Cobb 500 male broiler chicks was conducted to investigate the effects of feed containing short-chain fatty acid esters on the performance of coccidiosis vaccinated broilers. Day-of-hatch male Cobb 500 broiler chicks were obtained from Cobb-Vantress (Cleveland, GA). The test house was divided into pens of equal size, arranged along a central aisle. The birds were kept in 48 pens each having an area of 5×10=50 ft². All pens had approximately 4 inches of built-up litter with a coating of fresh pine shavings. The initial stocking density, after subtracting out for equipment, was ˜0.91 ft²/bird or 50 birds per pen. Each pen had 5 feet high side walls with bottom 1½ feet being of solid wood to prevent bird migration. All pens were numbered consecutively and identified on pen-cards. The diets were provided ad libitum in one tube-type feeder per pen. From day 1 until day 7, feed was also supplied on a tray placed on the litter of each pen. Water was provided ad libitum from one Ziggity nipple line per pen (six available nipples/pen).

Treatment feed and water were provided ad libitum from day 0 though day 42 of the study. The feed was provided ad libitum from the date of chick arrival (day 0) until termination (day 42) of the study. The diets were made for three growth phases, starter, grower and finisher (Tables 1 & 2). Unmedicated commercial type chicken rations were formulated with feedstuffs commonly used in the United States. Starter feed was fed from day 0 to day 21. Grower feed was fed from day 21 to day 35 and finisher feed was fed from day 35 to day 42. All diets were fed in pelleted or pelleted-crumbled form (starter diet). On day 21 and day 35 non-consumed feed was weighed by pen and new diets were issued. On day 42, non-consumed finished feed was weighed by pen and discarded. Birds were weighed by pen on day 21, day 35, and day 42.

TABLE 1
Basal diets for the Starter, Grower and Finisher phases.

| | Starter | Grower | Finisher |
|---|---|---|---|
| | (0 to 21 d) | (21 to 35 d) | (35 to 49 d) |
| | % | | |
| Ingredient Name | | | |
| CORN, YELLOW, GRAIN | 55.82 | 57.15 | 62.70 |
| SOYBEAN MEAL | 39.17 | 37.22 | 32.40 |
| DEHULLED, SOLVENT | | | |
| FAT, VEGETABLE | 1.78 | 2.87 | 2.72 |
| CALCIUM CARBONATE | 1.03 | 0.996 | 0.95 |
| DICALCIUM PHOSPHATE. | 0.98 | 0.81 | 0.58 |
| SALT, PLAIN (NaCl) | 0.39 | 0.39 | 0.24 |
| Methionine MHA | 0.38 | 0.30 | 0.22 |
| L-LYSINE | 0.21 | 0.1 | 0.04 |
| TRACE MINERAL PREMIX | 0.075 | 0.075 | 0.075 |
| VITAMIN PREMIX | 0.065 | 0.050 | 0.050 |
| L-Threonine 98.5 | 0.09 | 0.03 | 0.00 |
| Quantum Blu | 0.01 | 0.01 | 0.01 |
| Nutrient Name | | | |
| DRY MATTER | 85.74 | 85.75 | 85.36 |
| PROTEIN, CRUDE | 22 | 21 | 19.08 |
| FAT, CRUDE | 4.03 | 5.13 | 5.12 |
| FIBER, CRUDE | 2.21 | 2.17 | 2.14 |
| CALCIUM | 0.9 | 0.84 | 0.76 |
| PHOS. TOTAL | 0.57 | 0.53 | 0.47 |
| PHOS., AVAILABLE | 0.45 | 0.42 | 0.38 |
| M.E. POULTRY | 3,008.00 | 3,086.00 | 3,191.00 |
| METHIONINE | 0.63 | 0.56 | 0.47 |
| LYSINE | 1.29 | 1.16 | 1 |
| TRYPTOPHAN | 0.29 | 0.28 | 0.25 |
| THREONINE | 0.88 | 0.79 | 0.7 |
| SODIUM | 0.18 | 0.18 | 0.16 |
| POTASSIUM | 0.91 | 0.88 | 0.8 |
| CHLORIDE | 0.26 | 0.27 | 0.18 |
| dig methionine | 0.61 | 0.53 | 0.45 |
| dig cysteine | 0.25 | 0.25 | 0.23 |
| dig lysine | 1.18 | 1.05 | 0.9 |
| dig tryptophan | 0.28 | 0.27 | 0.24 |
| dig threonine | 0.78 | 0.69 | 0.61 |
| dig isoleucine | 0.86 | 0.83 | 0.75 |
| dig histidine | 0.5 | 0.48 | 0.45 |
| dig valine | 0.9 | 0.87 | 0.8 |
| dig leucine | 1.69 | 1.64 | 1.55 |
| dig arginine | 1.26 | 1.21 | 1.1 |
| dig phenylalanine | 1 | 0.96 | 0.89 |
| dig TSAA | 0.88 | 0.8 | 0.7 |

Calcium Minimum 3.20%, Maximum 4.20%; Iron 2.63%; Magnesium 2.68%; Manganese 13.40%; Zinc 10.70%; Copper 4000 ppm; Iodine 1000 ppm; Selenium 400 ppm.
²Vitamin A1,000,000 IU/lb; Vitamin D3 200,000 IU/lb; Vitamin E 2,000 IU/lb; Vitamin B-12 2.20 mg/lb; Riboflavin 800 mg/lb; Niacin 8,000 mg/lb; d-Pantothenic Acid 2,000 mg/lb; Choline 34,720 mg/lb; Menadione 132 mg/lb; Folic Acid 100 mg/lb; Thiamine 400 mg/lb; Pyridoxine 400 mg/lb; Biotin 20 mg/lb; Ethoxyquin 23,000 mg/lb

The treatments were replicated in 12 blocks and the 4 treatments were randomized within each block. The pen was the experimental unit. The doses used for each treatment are shown in Table 2. During diet manufacturing, the ester treatments were first mixed with the soybean oil, before adding to the mixer. Treatment 2 was made by adding the encapsulated calcium butyrate (Ca butyrate) directly to the mixer (ButiPEARL®, Kemin Industries Inc.).

TABLE 2
Treatments used in the performance trial.

| Treatment | Description |
|---|---|
| 1 | Control Diet |
| 2 | Control Diet + 500 g/MT Encapsulated Ca Butyrate |
| 3 | Control Diet + 322 g/MT Propylene Glycol Butyric |
| | Ester (C4-MPG)* |
| 4 | Control Diet + 375 g/MT Propylene Glycol Valeric |
| | Ester (C5-MPG)* |

*Organic acid dose calculated to be equal to the moles of organic acid in 500 g/MT of encapsulated Ca butyrate (2.38 mol of butyrate)

The statistical analysis and data graphs were done with the JMP v17.0 statistical software package (Cary, North Carolina). The data were analyzed by ANOVA, using Student's t-test for the analysis of means. Results are considered significant if P-values are ≤0.05.

HPLC analysis of propylene glycol esters (MPG esters). The percentage purity of valerate and butyrate MPG esters was determined using an Agilent 1260 Infinity II LC system with a diode array detector (DAD) at 210 nm. An aliquot of the reaction mixture was transferred to a HPLC vial and diluted 100 times with acetonitrile prior to injection. Peak separation was achieved with a Phenomenex Luna C18 (2) column (5 μm, 4.6 mm×250 mm) with column temperature maintained at 30° C. The mobile phase was set as 75% acetonitrile (Fisher Scientific; A998) and 25% Milli-Q deionized water with 0.2% phosphoric acid (Fisher Scientific; Spectrum Chemicals P1481), with a flow rate of 1.0 mL/min.

Establishing calibration curves to determine unreacted acid composition. The standard solutions were prepared by taking 1.412 g of butyric acid and 1 g of MPG in a 10 ml Falcon tube and 1.609 g valeric acid and 1 g MPG in another 10 ml Falcon tube. A standard curve of five different reference concentrations of this reaction mixture was prepared by making 1×, 0.8×, 0.6×, 0.4×, and 0.2× concentrations of these sample mixtures in acetonitrile. Two concentrations, 1× and 0.5× concentrations, were prepared with final ester products after synthesis in acetonitrile, as mentioned before. Based on the known reference concentrations of valeric and butyric acid in the calibration curves, the unreacted acids in the final product mixture were obtained.

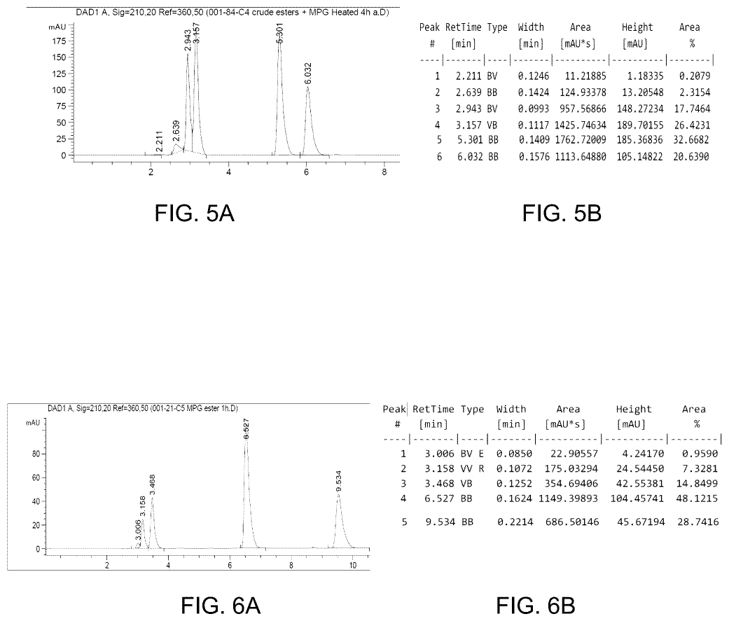

For the treatments used in Example 1, the final chromatogram of the propylene glycol butyrate esters and their area is shown in FIGS. 5A and 5B and a summary of the composition is given in Table 3. The final chromatogram of the valerate MPG esters and their area is shown in FIGS. 6A and 6B and a summary of the composition is given in Table 3. The calibration curves were established for the MPG valerate condition, and it was found that about 1.9% of unreacted valeric acid was present in the final product. Based on this number, the total esters were calculated to be 93% (mono-valerate 72% and di-valerate 21%), and unreacted MPG was 5%. The calibration curves were established for the MPG butyrate condition, and it was found that about 2.18% of unreacted butyric acid was present in the final product. Based on this number, the total esters were calculated to be 90% (mono-butyrate 68% and di-butyrate 22%) and unreacted MPG was 8%.

TABLE 3
Species composition and butyrate content of synthesized products.

| | | | |
|---|---|---|---|
| Material | Composition (%) | g/kg Product | Acid Content |

Propylene glycol butyric ester (C4-MPG)

| | | | |
|---|---|---|---|
| Mono-ester | 68 | 680 | 59.6% |
| Di-ester | 22 | 220 | 80.5% |
| Unreacted MPG | 8 | 80 | |
| Unreacted C4 acid | 2 | 20 | |
| Full mixture | | | 60.2% |

Propylene glycol valeric ester (C5-MPG)

| | | | |
|---|---|---|---|
| Mono-ester | 72 | 720 | 63.3% |
| Di-ester | 21 | 210 | 82.9% |
| Unreacted MPG | 5 | 50 | |
| Unreacted C5 acid | 2 | 20 | |
| Full mixture | | | 65% |

For the preparations used in Example 2, esterification reactions were optimized to produce mostly mono- or di-esters of butyric acid. These reactions underwent downstream processing to obtain the products that contain mostly mono- or di-esters of propylene glycol butyrate in the compositions (Table 4).

TABLE 4
Species composition and butyrate content
of the Mono and Di-MPG ester.

| | | |
|---|---|---|
| Material | Composition (%) | Acid Content |

Propylene glycol mono butyrate (mono C4-MPG)

| | | |
|---|---|---|
| Mono-ester | 83.15 | 59.6% |
| Di-ester | 10 | 80.5% |
| Unreacted C4 acid | 6.85 | 100% |
| Full mixture | | 64.4% |

Propylene glycol di butyrate (di C4-MPG)

| | | |
|---|---|---|
| Mono-ester | 10 | 59.6% |
| Di-ester | 82.3 | 80.5% |
| Unreacted C5 acid | 7.7 | 100% |
| Full mixture | | 79.9% |

The feed intake, feed conversion and average weight gain were calculated for days 21, 35, and 42. The values for the average feed conversion (FCR) at each feeding period are given in Table 5. The improvement in FCR was statistically significant for all the organic acid treatments at each time point compared to the negative control treatment.

TABLE 5
Average feed conversion ratio (FCR) during each phase of the trial.

| | FCR | | |
|---|---|---|---|
| Treatment | 0-21 d | 0-35 d | 0-42 d |
| 1) Control | 1.38 a | 1.55 a | 1.71 a |
| 2) Encapsulated Ca Butyrate | 1.30 b | 1.51 b | 1.63 b |
| 3) Propylene Glycol Butyrate Ester | 1.33 b | 1.51 b | 1.65 b |
| 4) Propylene Glycol Valerate Ester | 1.33 b | 1.51 b | 1.66 b |

Values with different letters are significantly different (P < 0.05).

The values for the average weight gain at each feeding period are given in Table 6. None of the improvements in weight gain for any of the treatments were statistically significant improvement. The values for the average feed intake at each feeding period are given in Table 7.

TABLE 6
Average weight gain during each phase of the trial.

| | Average Weight Gain (kg) | | |
|---|---|---|---|
| Treatment | 0-21 d | 0-35 d | 0-42 d |
| 1) Control | 0.701 a | 1.779 a | 2.456 a |
| 2) Encapsulated Ca Butyrate | 0.713 a | 1.819 a | 2.562 a |
| 3) Propylene Glycol Butyrate Ester | 0.700 a | 1.773 a | 2.499 a |
| 4) Propylene Glycol Valerate Ester | 0.699 a | 1.777 a | 2.496 a |

Values with different letters are significantly different (P < 0.05).

TABLE 7
Average feed intake per pen during each phase of the trial.

| | Average Feed Intake per pen (kg) | | |
|---|---|---|---|
| Treatment | 0-21 d | 0-35 d | 0-42 d |
| 1) Control | 50.69 a | 137.84 a | 205.92 a |
| 2) Encapsulated Ca Butyrate | 50.44 a | 136.11 ab | 202.21 a |
| 3) Propylene Glycol Butyrate Ester | 48.98 a | 133.05 b | 200.94 a |
| 4) Propylene Glycol Valerate Ester | 49.16 a | 135.70 ab | 205.08 a |

The resulting product was used in a broiler chicken performance trial, to determine if the esters have growth performance benefits. The propylene glycol esters are liquid and were added to the diet by mixing the correct amounts into the vegetable oil at room temperature before adding to the diet formulation in the mixer. The encapsulated Ca butyrate consists of calcium butyrate salt embedded in a wax-like hydrogenated vegetable oil matrix, which was added directly to the diet. The encapsulated Ca butyrate is known for the slow release of butyric acid in the intestinal tract at its site of action. When fed to animals, the butyric and valeric acid esters should remain intact until they reach the duodenum of the animal, where lipases will cleave the acids from propylene glycol. Therefore, when dosed at the same butyric acid level as the butyric acid content in 500 g/MT of encapsulated Ca butyrate, if the butyric acid is cleaved from the propylene glycol, then it shows the same performance benefits as the encapsulated Ca butyrate treatment.

The encapsulated Ca butyrate and ester treatments all improved the feed conversion ratio, as compared to the negative control, at all the timepoints measured (Table 5). The weight gain of the birds did not show statistically significant differences for any of the treatments (Table 6), but the encapsulated Ca butyrate and ester treatments showed numerical improvements in weight gain at 42 days. The treatments did not have large effects on feed intake, and the only statistically significant difference was with the propylene glycol butyrate ester at the 35-day measurement (Table 7). None of the treatments significantly affected the cumulative mortality of the birds (Overall mortality average of 10.7%).

The improvement in feed conversion observed with the ester treatments demonstrates that these molecules have the potential to be used as a feed additive in place of calcium butyrate. It is surprising that the propylene glycol valeric acid ester showed significant improvements in feed conversion at all timepoints. In general, valeric acid has shown antimicrobial activity, rather than growth-promoting activity. In butyric acid molar comparison, the propylene glycol ester treatments performed the same as the encapsulated Ca butyrate treatment.

### Example 2—Improved Growth Performance of Chickens Fed Mono or Di-Butyrate Esters of Propylene Glycol

A 42-day trial with 1,200 Cobb×Cobb 500 male broiler chicks was conducted to investigate the effects of feed containing mono- or di-butyric acid esters of propylene glycol on the performance of coccidiosis-vaccinated broilers. Day-of-hatch male Cobb 500 broiler chicks were obtained from Cobb-Vantress (Cleveland, GA). The test house was divided into pens of equal size, arranged along a central aisle. The birds were kept in 60 pens each having an area of 4.5×4.5=20.25 ft². All pens had approximately 4 inches of built-up litter with a coating of fresh pine shavings. The initial stocking density, after subtracting out for equipment, was ˜0.91 ft²/bird or 20 birds per pen. Each pen had 2 feet high side walls with bottom ½ foot being of solid wood to prevent bird migration. All pens were numbered consecutively and identified on pen-cards. The diets were provided ad libitum in one tube-type feeder per pen. From day 1 until day 7, feed was also supplied on a tray placed on the litter of each pen. Water was provided ad libitum from one Ziggity nipple line per pen.

Treatment feed and water were provided ad libitum from day 0 though day 42 of the study. The feed was provided ad libitum from the date of chick arrival (day 0) until termination (day 42) of the study. The diets were made for three growth phases, starter, grower and finisher (Table 8 & 9). Unmedicated commercial type chicken rations were formulated with feedstuffs commonly used in the United States. Pelleted-crumbled starter feed was fed from day 0 to day 21. Pelleted grower feed was fed from day 21 to day 35 and pelleted finisher feed was fed from day 35 to day 42. On day 21 and day 35 non-consumed feed was weighed by pen and new diets were issued. On day 42, non-consumed finished feed was weighed by pen and discarded. Birds were weighed by pen on day 21, day 35 and day 42.

TABLE 8
Basal diets for the Starter, Grower and Finisher phases.
Basal Diets

| | Starter | Grower | Finisher |
|---|---|---|---|
| | (0 to 21 d) | (21 to 35 d) | (35 to 49 d) |
| | % | | |
| Ingredient Name | | | |
| CORN, YELLOW, GRAIN | 55.82 | 57.15 | 62.70 |
| SOYBEAN MEAL | | | |
| DEHULLED, SOLVENT | 39.17 | 37.22 | 32.40 |
| FAT, VEGETABLE | 1.78 | 2.87 | 2.72 |
| CALCIUM CARBONATE | 1.03 | 0.996 | 0.95 |
| DICALCIUM PHOSPHATE. | 0.98 | 0.81 | 0.58 |
| SALT, PLAIN (NaCl) | 0.39 | 0.39 | 0.24 |
| Methionine MHA | 0.38 | 0.30 | 0.22 |
| L-LYSINE | 0.21 | 0.1 | 0.04 |
| TRACE MINERAL PREMIX | 0.075 | 0.075 | 0.075 |
| VITAMIN PREMIX | 0.065 | 0.050 | 0.050 |
| L-Threonine 98.5 | 0.09 | 0.03 | 0.00 |
| Quantum Blu | 0.01 | 0.01 | 0.01 |
| Nutrient Name | | | |
| DRY MATTER | 85.74 | 85.75 | 85.36 |
| PROTEIN, CRUDE | 22 | 21 | 19.08 |
| FAT, CRUDE | 4.03 | 5.13 | 5.12 |
| FIBER, CRUDE | 2.21 | 2.17 | 2.14 |
| CALCIUM | 0.9 | 0.84 | 0.76 |
| PHOS. TOTAL | 0.57 | 0.53 | 0.47 |
| PHOS., AVAILABLE | 0.45 | 0.42 | 0.38 |
| M.E. POULTRY | 3,008.00 | 3,086.00 | 3,191.00 |
| METHIONINE | 0.63 | 0.56 | 0.47 |
| LYSINE | 1.29 | 1.16 | 1 |
| TRYPTOPHAN | 0.29 | 0.28 | 0.25 |
| THREONINE | 0.88 | 0.79 | 0.7 |
| SODIUM | 0.18 | 0.18 | 0.16 |
| POTASSIUM | 0.91 | 0.88 | 0.8 |
| CHLORIDE | 0.26 | 0.27 | 0.18 |
| dig methionine | 0.61 | 0.53 | 0.45 |
| dig cysteine | 0.25 | 0.25 | 0.23 |
| dig lysine | 1.18 | 1.05 | 0.9 |
| dig tryptophan | 0.28 | 0.27 | 0.24 |
| dig threonine | 0.78 | 0.69 | 0.61 |
| dig isoleucine | 0.86 | 0.83 | 0.75 |
| dig histidine | 0.5 | 0.48 | 0.45 |
| dig valine | 0.9 | 0.87 | 0.8 |
| dig leucine | 1.69 | 1.64 | 1.55 |
| dig arginine | 1.26 | 1.21 | 1.1 |
| dig phenylalanine | 1 | 0.96 | 0.89 |
| dig TSAA | 0.88 | 0.8 | 0.7 |

Calcium Minimum 3.20%, Maximum 4.20%; Iron 2.63%; Magnesium 2.68%; Manganese 13.40%; Zinc 10.70%; Copper 4000 ppm; Iodine 1000 ppm; Selenium 400 ppm.
²Vitamin A1,000,000 IU/lb; Vitamin D3 200,000 IU/lb; Vitamin E 2,000 IU/lb; Vitamin B-12 2.20 mg/lb; Riboflavin 800 mg/lb; Niacin 8,000 mg/lb; d-Pantothenic Acid 2,000 mg/lb; Choline 34,720 mg/lb; Menadione 132 mg/lb; Folic Acid 100 mg/lb; Thiamine 400 mg/lb; Pyridoxine 400 mg/lb; Biotin 20 mg/lb; Ethoxyquin 23,000 mg/lb

The treatments were replicated in 12 blocks and the 5 treatments were randomized within each block. The pen was the experimental unit. The doses used for each treatment are shown in Table 9. During diet manufacturing, the ester treatments were first mixed with the soybean oil, before adding to the mixer. Treatment 2 was made by adding the encapsulated calcium butyrate directly to the mixer (ButiPEARL®, Kemin Industries Inc.).

TABLE 9
Treatments used in the performance trial

| Treatment | Description |
|---|---|
| 1 | Control Diet |
| 2 | Control Diet + 500 g/MT Encapsulated Ca Butyrate |
| 3 | Control Diet + 322 g/MT Propylene Glycol Monobutyrate* |
| 4 | Control Diet + 260 g/MT Propylene Glycol Dibutyrate* |
| 5 | Control Diet + 240 g/MT Tributyrin* |

*Butyric acid doses for the ester treatments calculated to be equal to the moles of butyric acid in 500 g/MT of encapsulated Ca butyrate (2.38 mol of butyrate)

The statistical analysis and data graphs were done with the JMP v17.0 statistical software package (Cary, North Carolina). The data were analyzed by ANOVA, using Student's t-test for the analysis of means. Results are considered significant if P-values are ≤0.05. The feed intake, feed conversion and average weight gain were calculated for days 21, 35 and 42.

The values for the average feed conversion (FCR) at each feeding period are given in Table 10. The improvement in FCR was statistically significant, as compared to the negative control treatment, for all of the butyrate treatments at the 35-day timepoint. At 42 days, the Encapsulated Ca butyrate, Propylene Glycol Monobutyrate and Tributyrin treatments had improved FCR that was statistically different from the control treatment, while the Propylene Glycol Dibutyrate treatment was intermediate.

TABLE 10
Average feed conversion ratio (FCR) during each phase of the trial

| | FCR | | |
|---|---|---|---|
| Treatment | 0-21 d | 0-35 d | 0-42 d |
| 1) Control | 1.40 a | 1.48 a | 1.58 a |
| 2) Encapsulated Ca Butyrate | 1.36 a | 1.42 b | 1.55 b |
| 3) Propylene Glycol Monobutyrate | 1.35 a | 1.43 b | 1.55 b |
| 4) Propylene Glycol Dibutyrate | 1.37 a | 1.45 b | 1.56 ab |
| 5) Tributyrin | 1.37 a | 1.45 b | 1.55 b |

Values with different letters are significantly different (P < 0.05)

The values for the average weight gain at each feeding period are given in Table 11. The improvement in weight gain for the Propylene Glycol Monobutyrate treatment was significantly improved as compared to the control treatment at each of the timepoints. At 35 and 42 days the average weight gain for both of the propylene glycol ester treatments was significantly improved as compared to the control treatment.

TABLE 11
Average weight gain during each phase of the trial.

| | Average Weight Gain (kg) | | |
|---|---|---|---|
| Treatment | 0-21 d | 0-35 d | 0-42 d |
| 1) Control | 0.60 b | 1.75 b | 2.42 b |
| 2) Encapsulated Ca Butyrate | 0.62 ab | 1.83 ab | 2.52 ab |
| 3) Propylene Glycol Monobutyrate | 0.66 a | 1.89 a | 2.64 a |
| 4) Propylene Glycol Dibutyrate | 0.65 ab | 1.85 a | 2.57 a |
| 5) Tributyrin | 0.64 ab | 1.82 ab | 2.52 ab |

Values with different letters are significantly different (P < 0.05).

The values for the average feed intake at each feeding period are given in Table 12. The feed intake for the Propylene Glycol Monobutyrate treatment was significantly higher than the control treatment at 35 and 42 days, while the other treatments were intermediate.

TABLE 12
Average feed intake per pen during each phase of the trial.

| | Average Feed Intake per pen (kg) | | |
|---|---|---|---|
| Treatment | 0-21 d | 0-35 d | 0-42 d |
| 1) Control | 17.72 a | 50.48 b | 74.64 b |
| 2) Encapsulated Ca Butyrate | 17.61 a | 51.15 ab | 76.73 ab |
| 3) Propylene Glycol Monobutyrate | 18.77 a | 53.80 a | 80.58 a |
| 4) Propylene Glycol Dibutyrate | 18.63 a | 52.79 ab | 78.55 ab |
| 5) Tributyrin | 18.16 a | 51.74 ab | 77.59 ab |

Values with different letters are significantly different (P < 0.05).

All of the treatments containing butyric acid, except for the Propylene Glycol Dibutyrate treatment, were statistically different from the negative control treatment at the 42-day timepoint (Table 10). None of the treatments significantly affected the cumulative mortality of the birds (6% average for the combined treatments). Surprisingly, Propylene Glycol Monobutyrate is the only treatment that is significantly better than the negative control diet for FCR, feed intake and average weight gain at both 35 and 42 days (Table 10, Table 11, and Table 12).

The improvement in feed conversion, weight gain and feed intake that was observed with the Propylene Glycol Monobutyrate treatment demonstrates that the monobutyrate form of the ester shows the best efficacy when fed to broiler chickens. In general, when esterified or encapsulated sources of butyric acid are fed at the same molar amount of butyrate acid similar efficacy results are seen. The fact that the monobutyrate form of the propylene glycol ester had better performance, as compared to the negative control treatment, was an unexpected result.

The equivalent efficacy of the MPG esters allows for lower inclusion in the feed to meet the same butyric acid levels, and a smaller carbon footprint to produce the butyric acid esters leads to a more sustainable product.

### Example 3—Effects of Supplementation Level of a Mixture of Mono and Di-Propylene Glycol on the Growth Performance of Broiler Chickens

A 42-day trial with 3,240 Ross 708 male broiler chicks was conducted to investigate the effects of feed containing different doses of a liquid propylene glycol-butyrate esters (30% monopropylene glycol butyrate, 70% di-propylene glycol butyrate) on the performance of broilers. Day-of-hatch male Ross 708 broiler chicks were obtained from Aviagen Hatchery in Blairsville, GA. At the hatchery, the birds were sexed and received routine vaccinations. The coccidia vaccine, Coccivac-B52, was applied on the birds upon arrival. The test house was divided into two rooms, each with 36 pens of equal size, arranged along a central aisle. The birds were kept in 72 pens that all had approximately 4 inches of built-up litter with a coating of fresh pine shavings. The initial stocking density, after subtracting out for equipment, was ˜0.73 ft²/bird. Each pen had 5 feet high side walls with a bottom 1½ feet being solid wood to prevent bird migration. All pens were numbered consecutively and identified on pen-cards. The diets were provided ad libitum in one tube-type feeder per pen. From day 1 until day 7, feed was also supplied on a tray placed on the litter of each pen. Water was provided ad libitum from one Ziggity nipple line per pen (six available nipples/pen).

Treatment feed and water were provided ad libitum from the date of chick arrival (day 0) until termination (day 42) of the study. The diets were made for three growth phases, starter, grower and finisher (Table 13). Unmedicated commercial type chicken rations were formulated with feedstuffs commonly used in the United States. Starter feed was fed from day 0 to day 21. Grower feed was fed from day 21 to day 35 and finisher feed was fed from day 35 to day 42. All diets were fed in pelleted or pelleted-crumbled form (starter diet). On day 21 and day 35 non-consumed feed was weighed by pen and new diets were issued. On day 42, non-consumed finisher feed was weighed by pen and discarded. Birds were weighed by pen on day 21, day 35, and day 42.

TABLE 13
Basal diets for the Starter, Grower and Finisher phases.

| | Starter | Grower | Finisher |
|---|---|---|---|
| | (0 to 21 d) | (21 to 35 d) | (35 to 49 d) |
| | % | | |
| Ingredient Name | | | |
| CORN, YELLOW, GRAIN | 58.96 | 63.92 | 68.21 |
| SOYBEAN MEAL | 34.86 | 29.88 | 25.85 |
| FAT, POULTRY | 1.83 | 2.35 | 2.51 |
| CALCIUM CARBONATE | 1.14 | 1.05 | 0.96 |
| DICALCIUM | 1.11 | 0.83 | 0.55 |
| PHOSPHATE. | | | |
| DL - METHIONINE | 0.43 | 0.38 | 0.37 |
| L - LYSINE | 0.43 | 0.41 | 0.43 |
| SODIUM BICARBONATE | 0.24 | 0.22 | 0.22 |
| L-THREONINE 98.5 | 0.23 | 0.18 | 0.17 |
| SALT, PLAIN (NaCl) | 0.22 | 0.24 | 0.24 |
| L-ARGININE | 0.17 | 0.16 | 0.18 |
| L-VALINE | 0.09 | 0.11 | 0.1 |
| L-ISOLEUCINE | 0.08 | 0.06 | 0.06 |
| TRACE MINERAL¹ | 0.075 | 0.075 | 0.08 |
| VITAMIN PREMIX² | 0.05 | 0.05 | 0.05 |
| HOSTAZYM X | 0.03 | 0.03 | 0.03 |
| QUANTUM BLUE | 0.01 | 0.01 | 0.01 |
| Nutrient Name | | | |
| DRY MATTER | 85.41 | 85.18 | 84.94 |
| PROTEIN, CRUDE | 21.74 | 19.64 | 18.06 |
| FAT, CRUDE | 4.12 | 4.74 | 4.98 |
| FIBER, CRUDE | 2.14 | 2.08 | 2.04 |
| CALCIUM | 0.9 | 0.8 | 0.7 |
| PHOS. TOTAL | 0.58 | 0.5 | 0.44 |
| PHOS., AVAILABLE | 0.45 | 0.4 | 0.35 |
| M.E. POULTRY | 3,007 | 3,067 | 3,104 |
| METHIONINE | 0.72 | 0.65 | 0.62 |
| LYSINE | 1.37 | 1.24 | 1.16 |
| TRYPTOPHAN | 0.26 | 0.23 | 0.21 |
| THREONINE | 0.95 | 0.84 | 0.78 |
| SODIUM | 0.18 | 0.18 | 0.18 |
| POTASSIUM | 0.84 | 0.76 | 0.69 |
| CHLORIDE | 0.25 | 0.25 | 0.25 |
| DIG METHIONINE | 0.69 | 0.63 | 0.6 |
| DIG CYSTEINE | 0.24 | 0.22 | 0.21 |
| DIG LYSINE | 1.26 | 1.14 | 1.07 |
| DIG TRYPTOPHAN | 0.26 | 0.23 | 0.21 |
| DIG THREONINE | 0.86 | 0.75 | 0.7 |
| DIG ISOLEUCINE | 0.86 | 0.76 | 0.7 |
| DIG HISTIDINE | 0.46 | 0.43 | 0.4 |
| DIG VALINE | 0.92 | 0.87 | 0.8 |
| DIG LEUCINE | 1.59 | 1.48 | 1.4 |
| DIG ARGININE | 1.32 | 1.2 | 1.12 |
| DIG PHENYLALANINE | 0.92 | 0.84 | 0.78 |
| DIG TSAA | 0.95 | 0.87 | 0.83 |

¹Calcium Minimum 3.20%, Maximum 4.20%; Iron 2.63%; Magnesium 2.68%; Manganese 13.40%; Zinc 10.70%; Copper 4000 ppm; Iodine 1000 ppm; Selenium 400 ppm.
²Vitamin A1,000,000 IU/lb; Vitamin D3 200,000 IU/lb; Vitamin E 2,000 IU/lb; Vitamin B-12 2.20 mg/lb; Riboflavin 800 mg/lb; Niacin 8,000 mg/lb; d-Pantothenic Acid 2,000 mg/lb; Choline 34,720 mg/lb; Menadione 132 mg/lb; Folic Acid 100 mg/lb; Thiamine 400 mg/lb; Pyridoxine 400 mg/lb; Biotin 20 mg/lb; Ethoxyquin 23,000 mg/lb

The 6 treatments were replicated in 12 blocks and the treatments were randomized within each block. The pen was the experimental unit. The doses used for each treatment are shown in Table 14. The propylene glycol-butyrate (MPG-Butyrate) esters used for the study contains a liquid mixture of 28% of two monoesters (propylene glycol and 2-butyrate2-hydroxypropyl butyrate), 67% dibutyrate, 1.7% monopropylene glycol, 2.3% butyric acid and 1% water. This liquid mixture of the propylene glycol-butyrate esters (65% by weight) was then sprayed onto silica (35% by weight) and fed as a dry product. During diet manufacturing, the treatments were added directly to the feed mixer. ButiPEARL® (Kemin Industries Inc.) was used as a source of encapsulated calcium butyrate. The statistical analyses were done with the JMP v17.0 statistical software package (Cary, North Carolina). The data were analyzed by ANOVA, using Student's t-test for the analysis of means. Results are considered significant if P-values are ≤0.05.

TABLE 14
Treatments used in the performance trial.

| Treat- | | Butyric | |
|---|---|---|---|
| ment | Description | acid dose | |
| 1 | Control Diet | | |
| 2 | Control Diet + 375 g/MT ButiPEARL | 154 | g/MT |
| 3 | Control Diet + 198 g/MT MPG-Butyrate esters | 77 | g/MT |
| 4 | Control Diet + 395 g/MT MPG-Butyrate esters | 154 | g/MT |
| 5 | Control Diet + 790 g/MT MPG-Butyrate esters | 308 | g/MT |
| 6 | Control Diet + 1185 g/MT MPG-Butyrate esters | 462 | g/MT |

The feed intake, feed conversion and average weight gain were calculated for days 21, 35, and 42. The values for the average feed conversion (FCR) at each feeding period are given in Table 15. The improvement in FCR was statistically significant for the 195 and 395 g/MT MPG-Butyrate Esters treatments at each time point compared to the negative control treatment. The FCR for the encapsulated calcium butyrate treatment was not statistically different from the MPG-butyrate ester treatments.

TABLE 15
Average feed conversion ratio (FCR) during each phase of the trial.

| | FCR | | |
|---|---|---|---|
| Treatment | 0-21 d | 0-35 d | 0-42 d |
| 1) Control | 1.398 a | 1.580 a | 1.662 a |
| 2) Encapsulated Ca Butyrate | 1.359 ab | 1.558 ab | 1.633 b |
| 3) 198 g/MT MPG-Butyrate Esters | 1.345 b | 1.542 b | 1.634 b |
| 4) 395 g/MT MPG-Butyrate Esters | 1.330 b | 1.541 b | 1.636 b |
| 5) 790 g/MT MPG-Butyrate Esters | 1.332 b | 1.570 a | 1.646 ab |
| 6) 1185 g/MT MPG-Butyrate Esters | 1.345 b | 1.567 ab | 1.637 b |

Values with different letters are significantly different (P < 0.05). A lower FCR value represents more efficient growth.

The values for the average weight gain at each feeding period are given in Table 16. At 21 days, weight gain for the MPG-Butyrate Esters treatments were significantly improved as compared to the control treatment for the 198, 395 and 790 g/MT doses. At 35 days significant differences from the Control treatment were seen with the 198 and 395 g/MT doses, but at 42 days none of the treatments were significantly different with regard to average weight gain. The average weight gain with the encapsulated calcium butyrate treatment was not statistically different from the MPG-butyrate ester treatments at any of the time points.

TABLE 16
Average weight gain during each phase of the trial.

| | Average Weight Gain (kg) | | |
|---|---|---|---|
| Treatment | 0-21 d | 0-35 d | 0-42 d |
| 1) Control | 0.662 b | 1.662 c | 2.328 a |
| 2) Encapsulated Ca Butyrate | 0.695 ab | 1.706 abc | 2.386 a |
| 3) 198 g/MT MPG-Butyrate Esters | 0.699 a | 1.727 ab | 2.384 a |
| 4) 395 g/MT MPG-Butyrate Esters | 0.708 a | 1.743 a | 2.394 a |
| 5) 790 g/MT MPG-Butyrate Esters | 0.698 a | 1.675 bc | 2.345 a |
| 6) 1185 g/MT MPG-Butyrate Esters | 0.693 ab | 1.686 abc | 2.369 a |

Values with different letters are significantly different (P < 0.05).

The values for the average feed intake at each feeding period are given in Table 17. There were no significant differences in feed intake between any of the treatments at each time point.

TABLE 17
Average feed intake per pen during each phase of the trial.

| | Average Feed Intake per pen (kg) | | |
|---|---|---|---|
| Treatment | 0-21 d | 0-35 d | 0-42 d |
| 1) Control | 41.27 a | 116.19 a | 168.65 a |
| 2) Encapsulated Ca Butyrate | 41.96 a | 117.27 a | 169.88 a |
| 3) 198 g/MT MPG-Butyrate Esters | 42.03 a | 118.45 a | 172.36 a |
| 4) 395 g/MT MPG-Butyrate Esters | 41.95 a | 119.27 a | 172.77 a |
| 5) 790 g/MT MPG-Butyrate Esters | 41.13 a | 115.51 a | 168.09 a |
| 6) 1185 g/MT MPG-Butyrate Esters | 41.51 a | 117.43 a | 170.79 a |

Values with different letters are significantly different (P < 0.05).

The trial was carried out to determine the dose of MPG-butyrate esters that yields the best growth performance benefits. Four doses of MPG-butyrate esters that differed by minimum 5-fold were fed to the birds. The doses were calculated based upon the amount of butyric acid in the encapsulated calcium butyrate control treatment (Table 14). The 198 g/MT, 395 g/MT and 790 g/MT doses improved average weight gain at 21 days and the 198 g/MT and 395 g/MT doses improved weight gain at 35 days. All of the MPG-butyrate esters treatments showed improved feed conversion at 42 days, where only the 198 g/MT, 395 g/MT, and 1185 g/MT treatments were significantly different from the Control treatment. Unexpectedly, the 198 g/MT dose of MPG-butyrate esters showed the same feed conversion as the encapsulated calcium butyrate treatment; compared to the encapsulated calcium butyrate, the 198 g/MT dose contains ½ of the butyric acid content.

The growth performance data show that feeding MPG-butyrate esters to broiler chickens can improve their growth performance, even at a butyric acid dose equal to half the dose in 375 g/MT of encapsulated calcium butyrate.

### Example 4—Comparison of Propylene Glycol-Butyrate Esters and Glycerol-Butyrate Esters on the Growth Performance and Carcass Characteristics of Broiler Chickens

A 35-day trial with 900 Ross 308 male broiler chicks was conducted to investigate the effects of different butyric acid ester products on the performance of broilers. The day-old Ross 308 chicks were spray vaccinated for Newcastle disease, infectious bronchitis, and infectious bursal disease on the day of hatch and further vaccinated intranasally for Newcastle disease and infectious bronchitis on day 10. The test house had two rows of 30 pens along the center of the concrete floor. The trial had 4 treatments, each with 15 replicate pens that contained 15 chicks. The walls of the pens were plastic mesh and the floor of each pen was covered with fresh rice hulls. Diets were provided ad libitum in one tube-type feeder per pen. For approximately the first week, feed was also supplied on a tray placed on the litter of each pen. Water was provided ad libitum from a gravity fed poultry water dispenser.

Treatment feed and water were provided from the date of chick arrival (day 0) until termination (day 35) of the study. The diets were made for three growth phases, starter, grower and finisher (Table 18). The coccidiostat (Salinomycin sodium, 60 ppm) was added in basal feed from feed mill up to the end of the grower period, day 24. Starter feed was fed from day 0 to day 10, grower feed was fed from day 11 to day 24 and finisher feed was fed from day 25 to day 35. All diets were fed in pelleted or pelleted-crumbled form (starter diet). On day 10 and day 25 non-consumed feed was weighed by pen and new diets were issued. On day 35, non-consumed finisher feed was weighed by pen. Birds were weighed by pen on day 10, day 24, and day 35. Mortalities were removed and recorded daily.

At the end of the 35-day trial, two birds per replicate were selected for the determination of carcass yield. The selected birds had body weights closest to the average body weight of the pen. The carcass parameters measured were: Live weight, slaughter weight, carcass weight, outer breast weight, thigh weight, drumstick weight, wing weight, abdominal fat weight and foot pad score. Intestinal samples were collected from one bird per pen from the duodenum, jejunum and ileum. The samples were fixed in paraffin and intestinal morphology were photographed by Canon EOS 700D under 40× magnification. Tarosoft Image Framework was used to measure the villus height, crypt depth and villus height to crypt depth ratio. Cecal contents from the sampled bird were collected for further analysis. The data were evaluated with ANOVA in a completely randomized design (CRD), using pen as the experimental unit.

The treatments were replicated in 15 blocks, and the 4 treatments were randomized within each block, with pen being the experimental unit for growth performance. The doses used for each treatment are shown in Table 19. The butyrate ester treatments were fed as dry products and were added directly to the feed in the mixer. The propylene glycol-butyrate (MPG-Butyrate) esters used for the study contains a liquid mixture of 28% of two monoesters (propylene glycol and 2-butyrate2-hydroxypropyl butyrate), 67% dibutyrate, 1.7% monopropylene glycol, 2.3% butyric acid and 1% water. This liquid mixture of the propylene glycol-butyrate esters (65% by weight) was then sprayed onto silica (35% by weight) and fed as a dry product.

TABLE 18
Basal diets for the Starter, Grower and Finisher phases.

| | Starter | Grower | Finisher |
|---|---|---|---|
| | (0 to 10 d) | (21 to 35 d) | (35 to 42 d) |
| Ingredient Name | % | | |
| Corn | 39.67 | 35.49 | 35.65 |
| Wheat | 10.00 | 20.00 | 25.00 |
| Palm oil | 3.89 | 1.50 | 1.26 |
| DDGS | 3.00 | 10.00 | 10.00 |
| Rice solvent bran | 3.00 | 3.16 | 4.72 |
| Soybean CP 48% | 32.03 | 21.99 | 15.87 |
| Rape seed | 3.00 | 4.00 | 4.00 |
| L-Lysine | 0.36 | 0.42 | 0.47 |
| DL-Methionine | 0.39 | 0.34 | 0.32 |
| L-Threonine | 0.15 | 0.14 | 0.15 |
| L-Valine | 0.12 | 0.13 | 0.16 |
| Mono-di-calcium | 2.16 | 1.65 | 1.35 |
| phosphate P 21 | | | |
| Calcium carbonate | 1.46 | 0.44 | 0.34 |
| Salt | 0.30 | 0.30 | 0.30 |
| Choline Chloride 60% | 0.28 | 0.27 | 0.25 |
| Vitamin/mineral premix | 0.18 | 0.18 | 0.18 |
| Total | 100 | 100 | 100 |

| Nutrient Name | Unit | | | |
|---|---|---|---|---|
| Metabolizable energy | Cal/kg | 2975 | 3050 | 3100 |
| for Poultry | | | | |
| Protein | % | 23.00 | 21.50 | 19.50 |
| Fat | % | 5.71 | 3.27 | 3.00 |
| Fiber | % | 4.10 | 4.23 | 4.18 |
| Calcium | % | 1.10 | 0.75 | 0.65 |
| Total Phosphorous | % | 0.93 | 0.88 | 0.81 |
| Avao;. Phosphate for | % | 0.50 | 0.42 | 0.36 |
| Poultry | | | | |
| Salt | % | 0.354 | 0.34 | 0.33 |
| Lysine | % | 1.43 | 1.28 | 1.17 |
| Methionine + Cysteine | % | 1.05 | 0.93 | 0.84 |
| Methionine | % | 0.71 | 0.64 | 0.60 |
| Threonine | % | 0.98 | 0.89 | 0.81 |
| Tryptophan | % | 0.27 | 0.24 | 0.20 |
| Valine | % | 1.14 | 1.04 | 0.96 |
| Choline | mg/Kg | 1700 | 1600 | 1500 |
| Lysine digestible | % | 1.32 | 1.18 | 1.08 |
| Methionine digestible | % | 0.683 | 0.61 | 0.58 |
| Methionine + Cysteine | % | 1.00 | 0.92 | 0.86 |
| digestible | | | | |
| Threonine digestible | % | 0.88 | 0.79 | 0.72 |
| Tryptophan digestible | % | 0.25 | 0.21 | 0.18 |
| Valine digestible | % | 1.00 | 0.91 | 0.84 |

TABLE 19
Treatments used in the performance trial.

| Treatment | Description |
|---|---|
| 1 | Control Diet |
| 2 | Control Diet + 325 g/MT MPG-Butyrate Esters |
| 3 | Control Diet + 325 g/MT Butyrate Glycerides |
| | Commercial Product |
| 4 | Control Diet + 325 g/MT Tributyrin |
| | Commercial Product |

TABLE 20
Average feed conversion ratio (FCR) during each phase of the trial.

| | FCR | | |
|---|---|---|---|
| Treatment | 1-10 d | 1-24 d | 1-35 d |
| 1) Control | 1.01 ± 0.02 a | 1.23 ± 0.03 a | 1.46 ± 0.02 a |
| 2) 325 g/MT MPG- | 1.01 ± 0.02 ab | 1.21 ± 0.02 a | 1.42 ± 0.03 a |
| Butyrate Esters | | | |
| 3) 325 g/MT Butyrate | 0.99 ± 0.02 b | 1.20 ± 0.03 a | 1.43 ± 0.04 a |
| Glycerides Product | | | |
| 4) 325 g/MT | 0.99 ± 0.02 b | 1.22 ± 0.02 a | 1.44 ± 0.05 a |
| Tributyrin Product | | | |

Values with different letters are significantly different (P < 0.05). A lower FCR value represents more efficient growth.

TABLE 21
Average weight gain during each phase of the trial.

| | Average Weight Gain (g) | | |
|---|---|---|---|
| Treatment | 1-10 d | 1-24 d | 1-35 d |
| 1) Control | 292.4 ± 4.13 ab | 1426.8 ± 25.3 a | 2566.2 ± 41.6 a |
| 2) 325 g/MT MPG-Butyrate | 294.4 ± 6.8 a | 1440.6 ± 25.9 a | 2604.7 ± 39.1 a |
| Esters | | | |
| 3) 325 g/MT Butyrate | 289.2 ± 7.7 b | 1428.13 ± 30.1 a | 2585.9 ± 54.7 a |
| Glycerides Product | | | |
| 4) 325 g/MT Tributyrin | 290.9 ± 7.8 ab | 1422.9 ± 27.3 a | 2575.5 ± 70.1 a |
| Product | | | |

Values with different letters are significantly different (P < 0.05).

TABLE 22
Average feed intake per pen during each phase of the trial.

| | Average Feed Intake per Pen (g) | | |
|---|---|---|---|
| Treatment | 1-10 d | 1-24 d | 1-35 d |
| 1) Control | 292.4 ± 4.1 a | 1751.8 ± 27.7 a | 3733.6 ± 46.7 a |
| 2) 325 g/MT MPG-Butyrate Esters | 295.0 ± 5.1 a | 1743.6 ± 17.4 ab | 3722.6 ± 60.0 a |
| 3) 325 g/MT Butyrate Glycerides | 286.5 ± 7.6 b | 1726.4 ± 40.4 b | 3693.4 ± 130.5 a |
| Product | | | |
| 4) 325 g/MT Tributyrin Product | 287.9 ± 6.0 b | 1734.3 ± 33.0 ab | 3707.5 ± 182.5 a |

Values with different letters are significantly different (P < 0.05).

TABLE 23
Total mortality and Return on Investment

| | | Return on |
|---|---|---|
| Treatment | Mortality % | Investment |
| 1) Control | 2.67 ± 4.2 a | 2.14 ± 4.20 b |
| 2) 325 g/MT MPG-Butyrate Esters | 0.44 ± 1.5 a | 5.64 ± 2.33 a |
| 3) 325 g/MT Butyrate Glycerides | 3.11 ± 4.27 a | 3.26 ± 3.26 ab |
| Product | | |
| 4) 325 g/MT Tributyrin Product | 3.65 ± 6.40 a | 2.09 ± 6.39 b |

Values with different letters are significantly different (P < 0.05).

The performance data at 35 days show that the average feed conversion and average weight gain for butyrate ester products are numerically better than the control treatment, but are not statistically different (Table 20 and Table 21). Feed intake was not influenced by dietary treatments (Table 22). The MPG-butyrate esters treatment had the lowest mortality of all the treatments (Table 23). The combination of lower mortality and improved feed conversion lead to a better return on investment for the MPG-butyrate esters treatment (Table 23).

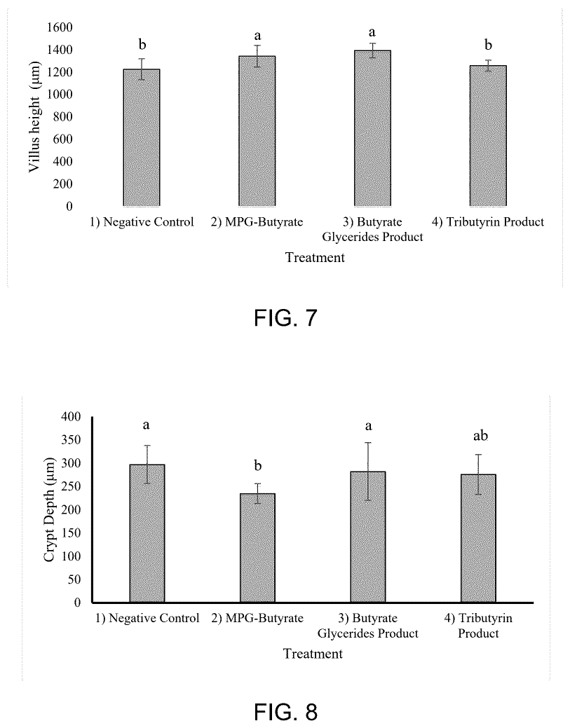

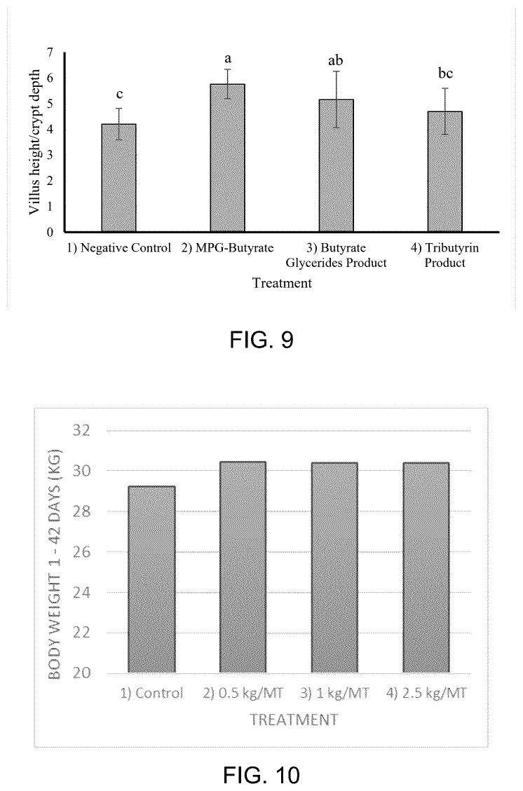

At the end of the 35-day trial, the birds fed the MPG-butyrate esters treatment had statistically higher slaughter weights compared to the tributyrin product treatment (Table 24). The MPG-butyrate esters treatment also had higher wing weight and drumstick weight as compared to the Butyrate Glycerides product and Tributyrin product treatments (Table 24). Histology analysis of intestinal samples showed that the MPG-butyrate esters treatment improved the villus height, crypt depth and villus height/crypt depth ratio of the duodenum samples (FIGS. 7-9). Overall, the MPG-Butyrate Esters treatment improved the performance parameters of weight gain, mortality, and ROI. Feeding MPG-Butyrate esters also improves the carcass characteristics of slaughter weight, wing weight and drumstick weight.

TABLE 24
Carcass characteristics

| Treatment | Slaughter wt.(g) | Wing wt. (g) | Drumstick wt. (g) |
|---|---|---|---|
| 1) Control | 2444.9 ± 113.7 ab | 192.8 ± 12.8 ab | 249.4 ± 15.6 b |
| 2) 325 g/MT MPG-Butyrate Esters | 2464.5 ± 67.6 a | 198.5 ± 10.0 a | 261.3 ± 13.6 a |
| 3) 325 g/MT Butyrate Glycerides | 2431.4 ± 89.3 ab | 191.3 ± 11.9 b | 246.3 ± 14.4 b |
| Product | | | |
| 4) 325 g/MT Tributyrin Product | 2412.9 ± 104.8 b | 190.4 ± 12.9 b | 249.5 ± 20.2 b |

Values with different letters are significantly different (P < 0.05).

### Example 5—Improved Growth Performance of Pigs Fed Propylene Glycol Esters of Butyric Acid

A 42-day trial was conducted to investigate the effects of MPG-butyrate esters on the growth performance of weaned pigs. The trial consisted of 128 weaned commercial crossbred piglets (Duroc×Large White×Landrace), castrated males and females with 8.5-9.5 kg body weight. The pigs were allocated to four treatments, and each treatment consisted of eight pens, each containing four pigs. These pens were further subdivided by sex: 4 pens for castrated male pigs and 4 pens for female pigs were assigned to each treatment. The average body weight of each pen was homogenized and balanced. The trial was divided into 2 phases: pre-starter I, 1-14 days, and Pre-starter II, 15-42 days. Feed was offered twice daily (7 am and 4 pm), and water was provided ad libitum via water nipples. The barn was cleaned every two days during the feeding trial, and pig feces were removed daily. The feed was fed in pelleted form (3-mm diameter pellets). The pelleting temperature was ˜75° C., and then the material was dried and cooled to an average temperature of 37° C., with a variation of ±5° C. from the ambient temperature. Basal diets were formulated to provide the same amount of nutrients and meet recommended commercial nutrition requirements.

TABLE 25
Diet composition

| | Pre-Starter Feed I | Pre-Starter Feed II |
|---|---|---|
| Item | Inclusion (%) | Inclusion (%) |
| Corn | 20.8 | 22.77 |
| Broken Rice | 20 | 10 |
| Cassava | 15 | 25 |
| Rice bran oil | 1 | 1 |
| SBM 48% CP | 29.1 | 31.89 |
| Soybean Full Fat | 5 | 3 |
| DL-Methionine | 0.14 | 0.16 |
| L-Lysine HCL | 0.37 | 0.38 |
| L-Threonine | 0.15 | 0.17 |
| Calcium Carbonate | 0.73 | 0.68 |
| MCP 22% | 1.08 | 1.16 |
| Salt | 0.35 | 0.39 |
| Choline Chloride 60% | 0.04 | 0.04 |
| Vitamin & Mineral | 0.5 | 0.5 |
| Premix | | |
| Whey (Lactose 70%) | 5.71 | 2.86 |
| NSP enzyme | 0.01 | 0.01 |
| Phytase | 0.02 | 0.02 |
| Total | 100 | 100 |

TABLE 26
Calculated nutrient contents:

| Nutrients | | |
|---|---|---|
| ME. For Swine (Kcal/kg) | 3,325.00 | 3,325.00 |
| Protein (%) | 21 | 21 |
| Fat (%) | 3.32 | 3.04 |
| Fiber (%) | 2.66 | 3.01 |
| Calcium (%) | 0.83 | 0.83 |
| Total Phosphorus (%) | 0.74 | 0.74 |
| Avail. P for Swine (%) | 0.45 | 0.45 |
| Salt (%) | 0.45 | 0.45 |
| Lysine (%) | 1.45 | 1.45 |
| Methionine + Cystine (%) | 0.45 | 0.8 |
| Methionine (%) | 0.8 | 0.46 |
| Tryptophan (%) | 0.28 | 0.28 |
| Threonine (%) | 0.97 | 0.97 |
| Choline (%) | 220 | 220 |
| Lactose (%) | 4 | 2 |

The trial treatments are shown in Table 27.

TABLE 27
Treatments used in the performance trial.

| Treatment | Description |
|---|---|
| 1 | Control Diet |
| 2 | Control Diet + 0.5 kg/MT MPG-Butyrate Esters |
| 3 | Control Diet + 1.0 kg/MT MPG-Butyrate Esters |
| 4 | Control Diet + 2.5 kg/MT MPG-Butyrate Esters |

The pigs were individually weighed on days 7, 14, 28, and 42. Feed was weighed on days 7, 14, 28, and 42, and feed intake was calculated based on the total feed provided minus the total leftover feed. Feed cost per gain (FCG) was calculated using a cost of 3.63 USD/kg.

Mortality, abnormal clinical signs, and causes of mortality were determined daily.

TABLE 28
Average Feed Intake (kg/day)

| Treatment | 1-14 days | 1-28 days | 1-42 days |
|---|---|---|---|
| 1) Control | 0.27 ± 0.03 | 0.56 ± 0.05 | 1.04 ± 0.09 |
| 2) 0.5 kg/MT MPG- | 0.26 ± 0.04 | 0.57 ± 0.05 | 1.02 ± 0.09 |
| Butyrate Esters | | | |
| 3) 1.0 kg/MT MPG- | 0.25 ± 0.02 | 0.58 ± 0.05 | 1.01 ± 0.07 |
| Butyrate Esters | | | |
| 4) 2.5 kg/MT MPG- | 0.25 ± 0.25 | 0.60 ± 0.03 | 1.04 ± 0.06 |
| Butyrate Esters | | | |

TABLE 29
Average Weight Gain (kg)

| Treatment | 1-14 days | 1-28 days | 1-42 days |
|---|---|---|---|
| 1) Control | 2.85 ± 0.80 | 11.63 ± 1.90 | 19.86 ± 2.57 |
| 2) 0.5 kg/MT MPG- | 2.90 ± 0.87 | 12.13 ± 1.79 | 21.15 ± 2.67 |
| Butyrate Esters | | | |
| 3) 1.0 kg/MT MPG- | 3.22 ± 0.94 | 12.03 ± 1.79 | 21.09 ± 2.13 |
| Butyrate Esters | | | |
| 4) 2.5 kg/MT MPG- | 2.95 ± 0.84 | 12.17 ± 1.64 | 21.09 ± 2.68 |
| Butyrate Esters | | | |

TABLE 30
Body Weight (kg)

| Treatment | 1-14 days | 1-28 days | 1-42 days |
|---|---|---|---|
| 1) Control | 12.14 ± 1.66 | 20.82 ± 2.43 | 29.23 ± 3.05 |
| 2) 0.5 kg/MT MPG- | 12.05 ± 1.37 | 20.71 ± 4.57 | 30.44 ± 3.05 |
| Butyrate Esters | | | |
| 3) 1.0 kg/MT MPG- | 12.32 ± 1.66 | 21.27 ± 2.24 | 30.41 ± 2.63 |
| Butyrate Esters | | | |
| 4) 2.5 kg/MT MPG- | 12.17 ± 1.42 | 21.46 ± 2.33 | 30.40 ± 3.01 |
| Butyrate Esters | | | |

TABLE 31
Feed Conversion (FCR)

| Treatment | 1-14 days | 1-28 days | 1-42 days |
|---|---|---|---|
| 1) Control | 1.41 ± 0.13 | 1.40 ± 0.10 | 2.17 ± 0.17 |
| 2) 0.5 kg/MT MPG- | 1.28 ± 0.39 | 1.30 ± 0.13 | 2.04 ± 0.23 |
| Butyrate Esters | | | |
| 3) 1.0 kg/MT MPG- | 1.16 ± 0.16 | 1.34 ± 0.09 | 2.02 ± 0.15 |
| Butyrate Esters | | | |
| 4) 2.5 kg/MT MPG- | 1.26 ± 0.19 | 1.38 ± 0.07 | 2.10 ± 0.16 |
| Butyrate Esters | | | |

TABLE 32
Feed Cost per Gain (FCG)

| Treatment | 1-14 days | 1-28 days | 1-42 days |
|---|---|---|---|
| 1) Control | 27.75 ± 2.62 | 25.19 ± 1.96 | 28.28 ± 1.81 |
| 2) 0.5 kg/MT MPG- | 25.40 ± 7.77 | 23.33 ± 2.94 | 27.43 ± 2.54 |
| Butyrate Esters | | | |
| 3) 1.0 kg/MT MPG- | 23.08 ± 3.12 | 24.95 ± 1.27 | 25.94 ± 2.31 |
| Butyrate Esters | | | |
| 4) 2.5 kg/MT MPG- | 25.28 ± 3.85 | 24.83 ± 0.53 | 26.93 ± 1.27 |
| Butyrate Esters | | | |

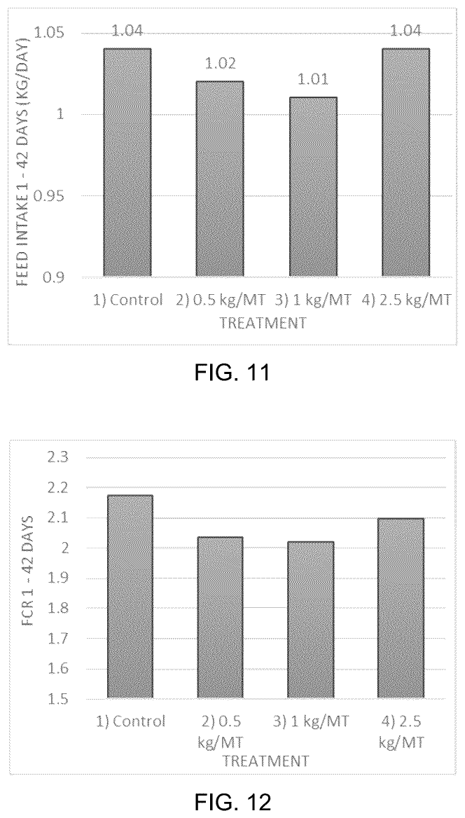

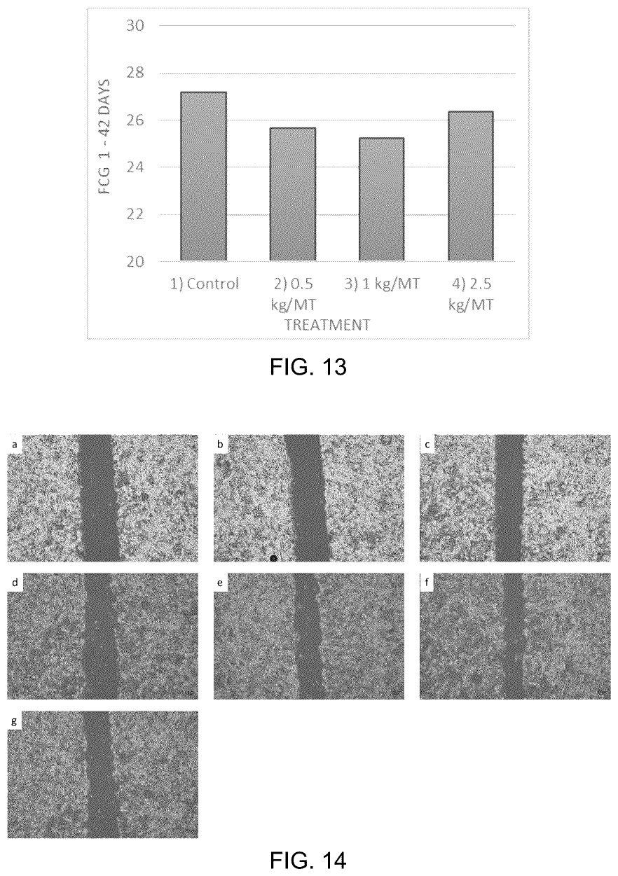

This data is also shown in FIGS. 10, 11, 12, and 13.

It can be concluded that, among the dietary treatments, pigs receiving 0.5 or 1 kg/MT feed of monopropylene glycol esters provided the best FCG, feed conversion, and improved average weight gain, which was compared to that of pigs fed the control diet. Thus, propylene glycol butyrate could be used as a feed additive to improve piglet production.

### Example 6—Evaluation of Propylene Glycol Valerate Ester for the Reduction of Mortality in Broiler Chickens Due to Necrotic Enteritis Caused by Clostridium perfringens

Necrotic Enteritis and Coccidiosis are major health issues related to reduced weight gain and decreased performance in broilers raised under antibiotic-free (ABF) conditions. Infection of broilers with Eimeria, specifically E. maxima, results in opportunistic growth of Clostridium perfringens in the gut, resulting in the development of Necrotic Enteritis. This study evaluated the ability of monopropylene glycol valerate ester to reduce the severity of intestinal lesions and incidence of mortality due to Necrotic Enteritis.

A 28-day study with male broiler chicks was conducted to investigate the ability of propylene glycol valerate esters (MPG-C5) to suppress the negative performance and intestinal health consequences of a C. perfringens infection. The composition of propylene glycol valerate ester used for the trial is described in Table 33.

TABLE 33
Composition of propylene valerate esters (MPG- C5)

| Entry | Composition | % mass |
|---|---|---|
| 1. | Propane-1,2-diyl dipentanoate | 68% |
| 2. | 2-hydroxypropyl pentanoate | 17.9% |
| 3. | 1-hydroxypropan-2-yl pentanoate | 8.4% |
| 4. | Propylene glycol and water | 4% |
| 5. | Valeric acid | 1.7% |

Day of hatch Cobb×Cobb 500 strain broiler chicks were obtained from Cobb-Vantress, Cleveland, Georgia, USA. The experiment was conducted in Petersime battery cages with floor space of 0.63 sq. ft/bird, and where the cage served as the experimental unit. Thermostatically controlled gas furnace/air conditioner maintained uniform temperature that was maintained at the appropriate temperature for the age of the birds and even illumination was provided. The experiment had 70 cages of 8 male broiler chickens per cage and the treatments were replicated in ten blocks; the seven treatments randomized within each block, resulting in 80 birds/treatment and a total of 560 birds in the trial.

A total of seven different treatments were tested in the challenge study (Table 34). All treatment groups were challenged with 5,000 oocysts/bird of Eimeria maxima by oral gavage on d14. On d19, 20, and 21 all birds, except treatment 1 birds, were orally inoculated with a fresh C. perfringens inoculum (1.0E8 cfu/mL) in a 1.0 mL oral gavage. A field isolate of C. perfringens known to cause necrotic enteritis and originating from a commercial broiler operation was utilized as the challenge organism.

TABLE 34
Treatments used in the challenge trial

| Treat- | |
|---|---|
| ment | Description |
| 1 | (Unchallenged control) Non-medicated Basal diet/not infected |
| 2 | (Challenged control) Non-medicated Basal diet/infected |
| 3 | (Positive control) Basal diet - medicated with BMD |
| | (50 g/t)/infected |
| 4 | Non-medicated Basal diet with MPG-C5 (0.5 kg/MT)/infected |
| 5 | Non medicated basal diet with MPG-C5 (1.0 kg/MT)/infected |
| 6 | Non medicated basal diet with MPG-C5 (2.0 kg/MT)/infected |
| 7 | Non medicated basal diet with MPG-C5 (3.0 kg/MT)/infected |

A non-medicated (no antibiotic growth promoter and no anticoccidial drug) corn-soybean based mash starter ration was fed during the study. The formulated diet used was the same as described in Table 35 and 36. The MPG-C5 treatment was mixed with the poultry fat before that ingredient was added to the diet. Bacitracin Methylene Disalicylate (BMD) at 50 g per ton was used as the positive control. The diet was pelleted, with a post-pelleting target moisture content of 13% and a PDI of 85%, and all feed was fed in the form of crumbled pellets. The rations were fed from the date of chick arrival until Day 28 of the study. Feed and water were available ad libitum throughout the trial. Birds found dead during the study were noted.

TABLE 35
Composition of the non-medicated diet
used in the Eimeria challenge trial.

| Ingredients | Percent (%) |
|---|---|
| Corn, yellow, grain | 59.02 |
| Soybean meal | 34.86 |
| Fat, Poultry | 1.83 |
| Calcium carbonate | 1.14 |
| Dicalcium phosphate | 1.11 |
| DL-Methionine | 0.43 |
| L-lysine | 0.43 |
| Sodium Bicarbonate | 0.24 |
| L-Threonine 98.5 | 0.23 |
| Salt, plain (NaCl) | 0.22 |
| L-Arginine | 0.17 |
| L-Valine | 0.09 |
| L-Isoleucine | 0.08 |
| Trace Mineral | 0.075 |
| Vitamin Premix | 0.05 |
| Hostazym X | 0.03 |
| Quantum Blue | 0.01 |

TABLE 36
Nutrient composition of the non-medicated diet
used in the Eimeria challenge trial.

| Nutrient | Amount (%) | Nutrient | Amount (%) |
|---|---|---|---|
| Dry matter | 85.41 | Dig methionine | 0.69 |
| Protein, crude | 21.74 | Dig cysteine | 0.24 |
| Fat, crude | 4.12 | Dig lysine | 1.26 |
| Fiber, crude | 2.14 | Dig tryptophan | 0.26 |
| Calcium | 0.9 | Dig threonine | 0.86 |
| Phos. Total | 0.58 | Dig isoleucine | 0.86 |
| Phos. Available | 0.45 | Dig histidine | 0.46 |
| M.E. Poultry (kcal/kg) | 3,007 | Dig valine | 0.92 |
| Methionine | 0.72 | Dig leucine | 1.59 |
| Lysine | 1.37 | Dig arginine | 1.32 |
| Tryptophan | 0.26 | Dig phenylalanine | 0.92 |
| Threonine | 0.95 | Dig TSAA* | 0.95 |
| Sodium | 0.18 | | |
| Potassium | 0.84 | | |
| Chloride | 0.25 | | |

Bird weight (kg) and feed consumption by cage were recorded at study initiation (d0), d14, d21, and termination (d28). The birds and feed were weighed by cage on d0, 14, 21, and 28. Means for cage weight gain (d0-14, 14-21, 14-28, 0-21, and 0-28), feed consumption, and feed conversion ratio (FCR) were then calculated. FCR was adjusted to account for mortality occurring during the study.

The response variables measured included growth performance, necrotic enteritis lesion scores, and mortality. On d21, three birds from each cage were selected, sacrificed, weighed, and examined for the presence of necrotic enteritis lesions. Lesion scores were determined by using the necrotic enteritis lesion scoring system, which is based on a 0 to 3 score, with 0 being no lesions, 1 being mild lesions, 2 being moderate lesions, and 3 being marked to severe lesions.

### Results

At 21 days, (i.e., 7 days after being challenged with coccidia and 3 days after being challenged with C. perfringens), the adjusted feed conversion rate (FCR) observed with MPG-C5 treatments (0.5-3.0 kg/MT; Day 14-21) and 0.5 kg/MT of MPG-C5 (Day 0-21) were significantly lower than the positive control (i.e., No additive added but is challenged) (Table 37). A significantly lower adjusted FCR was also observed with all MPG-C5 treatments at day 28 compared to the positive control. The lesion score for negative control (i.e., no treatment, no challenge) is the lowest, with the positive control (i.e., no treatment but challenged) being the highest. Among the treatments, MPG-C5 at 0.5 kg/MT had the lowest average lesion scores, which were significantly lower than those of the challenged control treatment (Table 37). In addition, at treatment of 1.0 kg/MT MPG-C5, the percent Necrotic Enteritis Mortality was significantly lower than the value for both the challenged control and the BMD treatments.

In summary, the FCR data demonstrate that the MPG-C5 treatments improve the growth performance of the birds. The lesion scoring and percent necrotic enteritis mortality data indicate that the MPG-C5 esters reduce the incidence of necrotic enteritis during challenges with both Eimeria maxima and C. perfringens. The study concludes that monopropylene glycol valerate ester can lessen the severity of intestinal lesions and incidence of mortality due to Necrotic Enteritis.

TABLE 37
Feed intake, weight gain, and adjusted feed conversion in birds treated with MPG-C5 or
bacitracin (BMD) and challenged with coccidiosis and Clostridium perfringens (CP).

| | | 1 | 2 | 3 | 4 | 5 | 6 | 7 |
|---|---|---|---|---|---|---|---|---|
| | | No | No | BMD, | MPG- | MPG- | MPG- | MPG- |
| Group | | Additive, | Additive, | 50 g/t, | C5, 0.5 | C5, 1.0 | C5, 2.0 | C5, 3.0 |
| Treatment | | No CP | CP | CP | kg/MT | kg/MT | kg/MT | kg/MT |
| Day | Feed intake | 3.687b | 3.674b | 4.083a | 3.714b | 3.732b | 3.701b | 3.670b |
| 14 | Adjusted | 1.348ab | 1.319ab | 1.295b | 1.319ab | 1.338ab | 1.382a | 1.355ab |
| | FCR | | | | | | | |
| | Weight gain | 0.304b | 0.308b | 0.356a | 0.312b | 0.315b | 0.295b | 0.299b |
| | (kg) | | | | | | | |
| Day | Feed intake | 4.507ab | 4.567ab | 4.627a | 4.434ab | 4.349ab | 4.272ab | 4.379b |
| 14-21 | Adjusted | 1.356c | 1.563a | 1.478ab | 1.439bc | 1.426bc | 1.416bc | 1.430bc |
| | Weight gain | 0.441a | 0.368c | 0.399b | 0.387bc | 0.387bc | 0.383bc | 0.385bc |
| | (kg) | | | | | | | |
| Day | Feed intake | 8.194ab | 8.241ab | 8.710a | 8.148ab | 8.081b | 7.973b | 8.049b |
| 0-21 | Adjusted | 1.425b | 1.529a | 1.462b | 1.462b | 1.465ab | 1.485ab | 1.478ab |
| | FCR | | | | | | | |
| | Weight gain | 0.746a | 0.676b | 0.756a | 0.699ab | 0.702ab | 0.678b | 0.684b |
| | (kg) | | | | | | | |
| Day | Feed intake | 11.469a | 11.077a | 11.425a | 10.955a | 11.214a | 10.973a | 11.113a |
| 0-28 | Adjusted | 1.402c | 1.550a | 1.487b | 1.458bc | 1.449bc | 1.479b | 1.483b |
| | FCR | | | | | | | |
| | Weight gain | 1.198a | 1.036b | 1.147ab | 1.133ab | 1.154ab | 1.108ab | 1.082ab |
| | (kg) | | | | | | | |
| Lesion | | 0.0d | 1.2a | 0.8ab | 0.5c | 0.9ab | 0.9ab | 0.6bc |
| scores | | | | | | | | |
| % NE | | 0.0c | 7.5ab | 8.8a | 10.0a | 1.3bc | 5.0abc | 5.0abc |
| Mortality | | | | | | | | |

Values with different alphabets in the same column are significantly different (p < 0.05).

### Example 7—the Effects of Monopropylene Valerate on the Recovery of Intestinal Barrier Using IPEC-J2 Cell Model

The effectiveness of monopropylene valerate (MPG-C5) on the intestinal barrier using the porcine intestinal epithelial cell line (IPEC-J2) monolayer model was studied. By creating a physical scratch and observing the wound closure, the results showed that MPG-C5 is effective in aiding intestinal barrier recovery and cell rehabilitation at a concentration as low as 0.1 mM. The IPEC-J2 monolayer was used as a model to mimic the intestinal barrier function of piglets, to investigate the effect of monopropylene valerate, MPG-C5, on intestinal barrier recovery following physical scratch-induced damage.

### Materials and Methods

Cell culture. IPEC-J2, a porcine intestinal epithelial cell-line isolated from mid jejunum, was cultured in Dulbecco's modified eagle medium/nutrient mixture (DMEM/F12; Corning Inc, USA) supplemented with 10% fetal bovine serum (FBS; Corning Inc, USA) and 1% antibiotic-antimycotic (Sigma-Aldrich, USA), at 37° C. in a 5% carbon dioxide (CO₂) incubator.

Maintenance of IPEC-J2 cells in a six-well plate. IPEC-J2 cells were seeded into a six-well plate (Corning Inc, USA) at a cell density of ca. 10⁶ cells/mL. Cells were cultured in DMEM/F12 (Corning Inc, USA) supplemented with 10% FBS and 1% antibiotic-antimycotic (Sigma-Aldrich, USA). The seeded cells were left to incubate for 48 hours at 37° C. and 5% CO₂ to reach 100% confluency before starting the assay.

Induced barrier damage by physical scratch (Trial 1). To study the effects of the treatments on the recovery of IPEC-J2 cells after injury, a scratch test was conducted. The test was conducted as previously described by Jiang et al. (2021) with some modifications. A 10 μL pipette tip was used to linearly scratch the IPEC-J2 cells, already confluent at 100%, in a six-well plate. Each well was washed thrice with phosphate-buffered saline (PBS; Cytiva Hyclone, USA) to remove the dislodged cells. Subsequently, 2 mL of the treatments, prepared in growth medium, were added into the wells in triplicate. Two mL of growth medium was added to the control wells. Valeric acid was first prepared at 100 mM and subsequently diluted in the growth medium to prepare 1.0 and 0.1 mM. MPG-C5, containing 44% valeric acid, was prepared at 100 mM valeric acid equivalent and subsequently diluted in growth medium to 1.0 and 0.1 mM for comparison. Each treatment was carried out in triplicate. The recovery of IPEC-J2 cells was observed based on the cell confluency in the well. The cell confluency is monitored visually at 0 and 4 hours using an inverted microscope.

Induced barrier damage by physical scratch (Trial 2). The test was conducted as previously described by Jiang et al. (2021) with some modifications. A 10 μL pipette tip was used to linearly scratch the IPEC-J2 cells, already confluent at 100%, in a six-well plate. Each well was washed thrice with phosphate-buffered saline (PBS; Cytiva Hyclone, USA) to remove the dislodged cells. Subsequently, 2 mL of the treatments, prepared in DMEM (low glucose; Corning Inc, USA) supplemented with 5% FBS, 1% antibiotic-antimycotic, and 1 ng/mL human epidermal growth factor (hEGF; Sigma-Aldrich, USA), were added to the wells in triplicate. Two mL of the same medium concoction (without any treatment) was added to the control wells. Valeric acid was first prepared at 100 mM and subsequently diluted in the growth medium to prepare 1.0 and 0.1 mM. MPG-C5, containing 44% valeric acid, was prepared at 100 mM valeric acid equivalent and subsequently diluted in the medium to 1.0 and 0.1 mM for comparison. Each treatment was carried out in triplicate. The recovery of IPEC-J2 cells was observed based on the cell confluency in the well. The cell confluency is monitored visually at 0 and 5 hours using an inverted microscope.

### Results

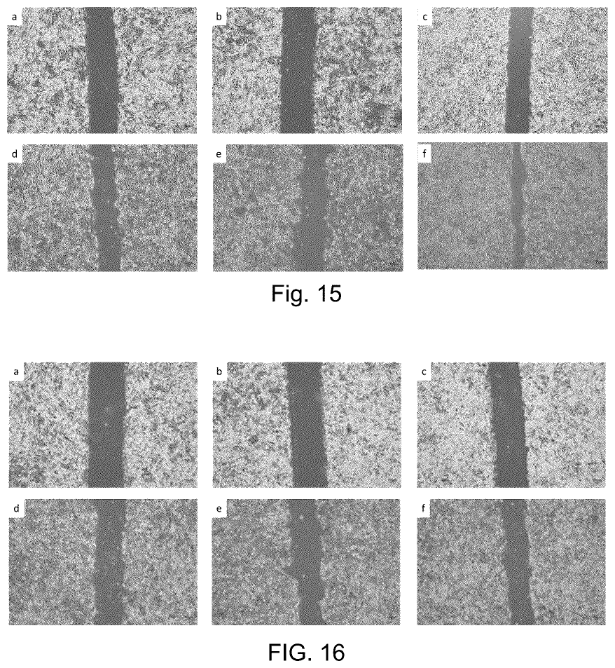

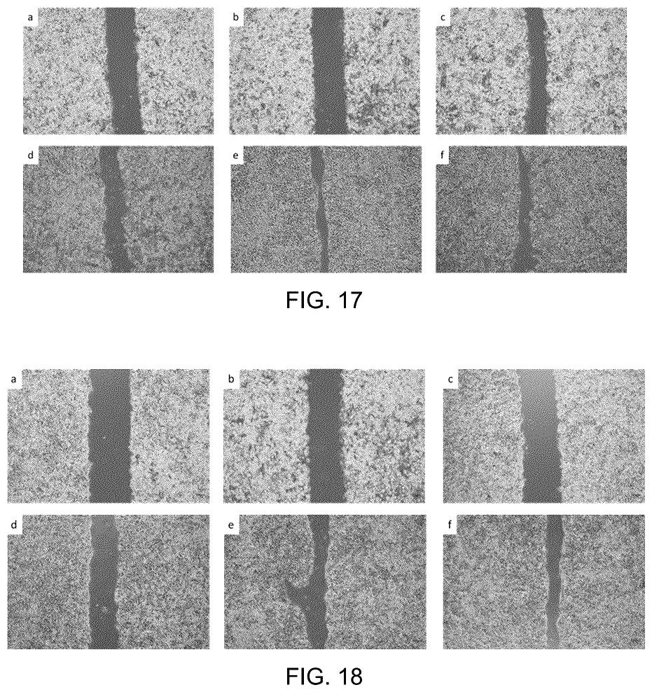

The effect of MPG-C5 on wound recovery (Trial 1). To study the effect of MPG-C5 on the recovery of IPEC-J2 monolayer, scratched cell monolayers were monitored over four hours. The effect was compared with that of valeric acid at the same molar concentrations, 0.1 and 1.0 mM. Without any treatment, it is evident that minimal recovery of the monolayer was observed (FIG. 14). With valeric acid and MPG-C5 added after the scratch, obvious wound closure can be observed where the vertical scratch was narrower after four hours of incubation (FIGS. 15-18).

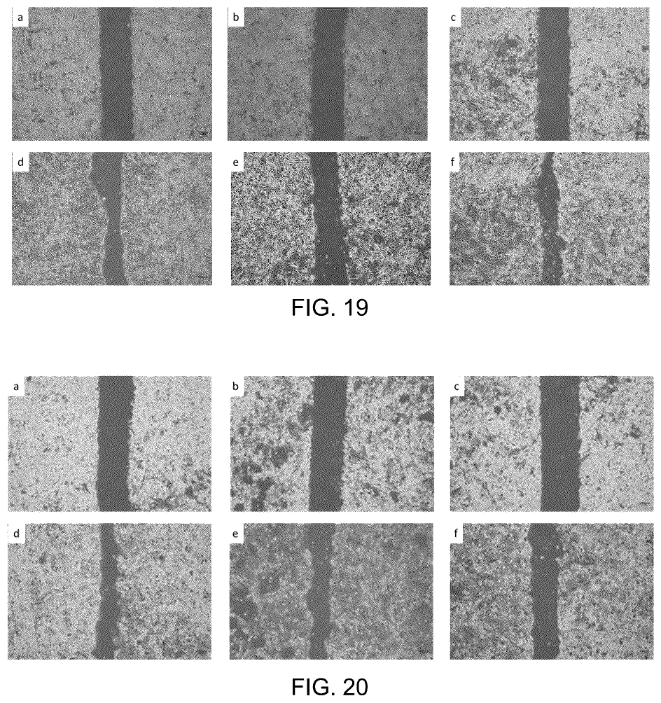

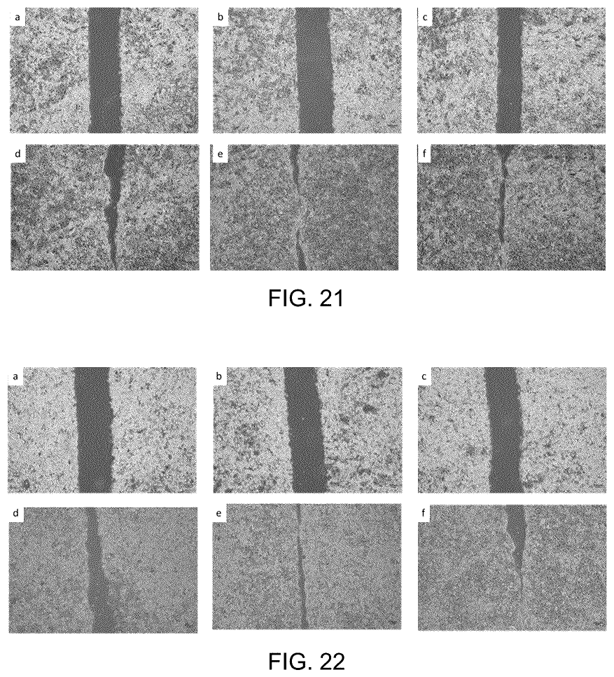

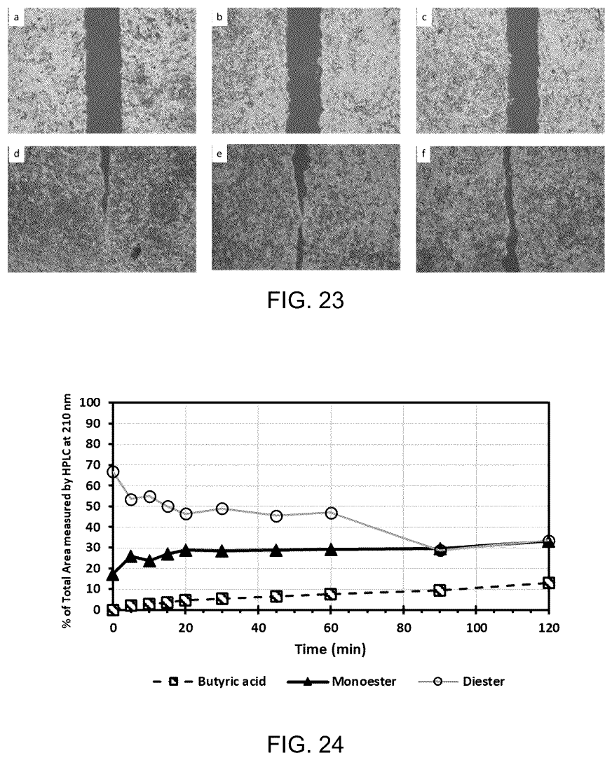

The effect of MPG-C5 on wound recovery (Trial 2). Following trial 1, a second trial was conducted with slight differences in the cell culturing conditions, and images were taken after five hours of incubation. In this trial, more wound closure was observed in the control compared to Trial 1 (FIG. 19). This could be due to the addition of a growth factor that encourages cell growth and proliferation. With the treatments, physical scratches were almost recovered in certain treated areas (FIG. 20 d-f, FIG. 21 d-f, FIG. 22 d-f, and FIG. 23 d-f).

### DISCUSSION

A healthy intestinal barrier is imperative as the first line of defense against infections. An impaired intestinal barrier (i.e., leaky gut) leads to increased intestinal permeability and encourages bacterial infection. On the other hand, a healthy intestinal barrier is positively correlated with growth performance. It is thus imperative to innovate and provide an effective feed additive to address this. The effectiveness of MPG-C5 in rehabilitating intestinal barrier function was evaluated.

The results suggest that treatments with monopropylene glycol valerate ester were effective in aiding cell recovery from physical scratches, with obvious wound closure observed in the treated areas. The effects of 1.0 mM valeric acid and 0.1 mM MPG-C5 were similar at the same treatment concentrations. It is thus noteworthy that MPG-C5 exerts beneficial roles in rehabilitating the monolayers after scratch damage.

### Example 8—the Effects of Pancreatic Lipase on the Antimicrobial Activity of Propylene Glycol-Butyrate Esters and Tributyrin

Triglycerides such as tributyrin do not have inherently high antimicrobial activity. In the intestines of animals and humans, pancreatic lipases are the enzymes mostly involved in cleaving fatty acids from dietary fat, but they also act on triglycerides with short-chain fatty acids. In vitro lipase digestion assays were carried out with MPG-butyrate and tributyrin. The inclusion of the MPG-butyrate and tributyrin in the assays was adjusted so that the total butyric acid amount in the assay was equal for the two products. During the lipase digestion time course, samples of the lipase assay solution were taken at different time points. Each timepoint sample was analyzed for lipase hydrolysis products and for antimicrobial activity against a hemolytic strain of E. coli.

For the lipase hydrolysis time course, 2.52 g of MPG-butyrate (72% butyric acid, 34. % monoesters, and 66. % diester by HPLC analysis, and 72% total butyric acid content) or 2.075 g of Tributyrin (>99% by HPLC analysis, T8626, Sigma Aldrich and 86% butyric acid content) was mixed with 47 ml of Phosphate Buffered Saline, PBS (10 mM Phosphate, pH 7.4, 137 mM NaCl, 2.7 mM KCl), in a 125 ml Erlenmeyer flask. A stir bar was added and a magnetic stir plate provided constant stirring during the reaction. A pH electrode was placed in the flask and the pH was adjusted to 7.4, if needed. The 0 min timepoint was collected before the addition of the enzyme. Lipase (1 ml of 33 mg/ml porcine pancreatic lipase (L3126, Sigma Aldrich) in PBS) was added to start the hydrolysis. During the time course, the assay solution was maintained at a pH of 7 by the addition of 2 M NaOH. Aliquots of 1 ml were taken periodically and placed in an 80° C. heat block for 2 minutes to inactivate the lipase, then placed on ice until analysis.

The inactivated samples were briefly vortexed, then aliquots of 100 μl were diluted into 900 μl of acetonitrile with 0.3% H₃PO₄ and the sample was analyzed by HPLC. The products of lipase hydrolysis were measured using an Agilent 1100 HPLC system with a diode array detector (DAD) at 210 nm. Peak separation was achieved with a Phenomenex Luna C18 (2) column (5 μm, 4.6 mm×150 mm) with the column temperature maintained at 30° C. The mobile phase was set as 65% acetonitrile (Fisher Scientific; A998) and 35% of 0.2% phosphoric acid (Fisher Scientific; Spectrum Chemicals P1481) in Milli-Q deionized water, at a flow rate of 1.0 mL/min. Analysis of the MPG-butyrate sample by base hydrolysis showed it to have a butyric acid content of 72%. Tributyrin has a butyric acid content of 86.4%. The concentration of butyric acid in the samples was determined using a standard curve made from diluting a mixture of 62% butyric acid and 38% MPG into acetonitrile (2-40 mM butyric acid).

Antimicrobial activity was measured in a 96 well plate format, using a Molecular Devices i3X plate reader and clear, flat bottom 96 well microtiter plates. All assayed samples were sterile filtered with a 0.22 mm syringe filter before plating. The sample wells contained 100 μl of lipase hydrolysis sample and 100 μl of bacterial culture. Control wells contained either PBS or uninoculated 2× TSB. A field strain of hemolytic E. coli, HuG, was grown in TSB Media for 20 hours, then counted on a hemocytometer and diluted to 1×10⁶ CFU/mL in 2× TSB media. The diluted E. coli was added to the wells (100 μl), then the plate was sealed with tape. The growth at 37° C. was measured by following the absorbance at 600 nm.

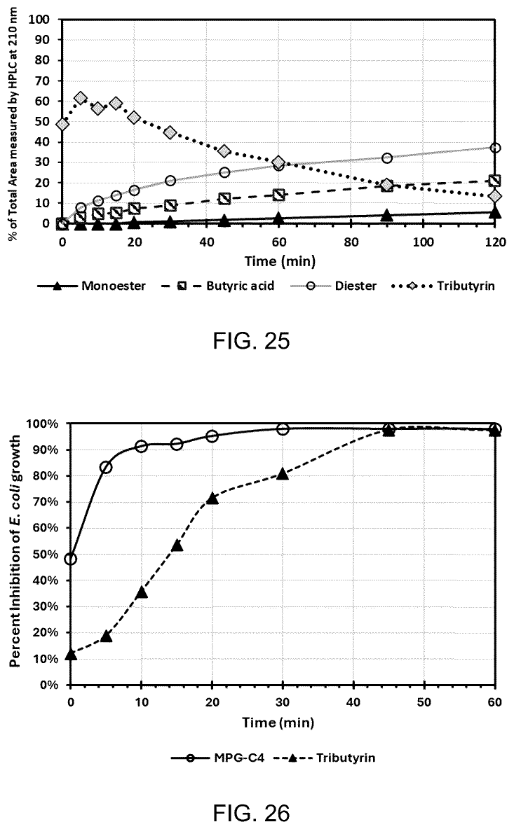

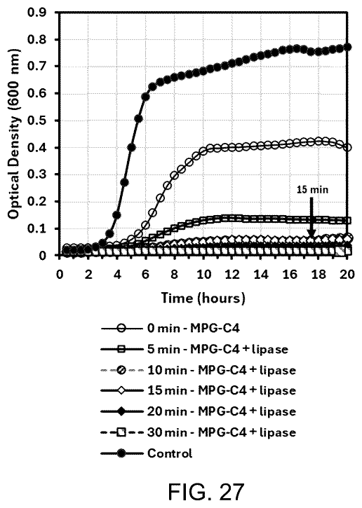

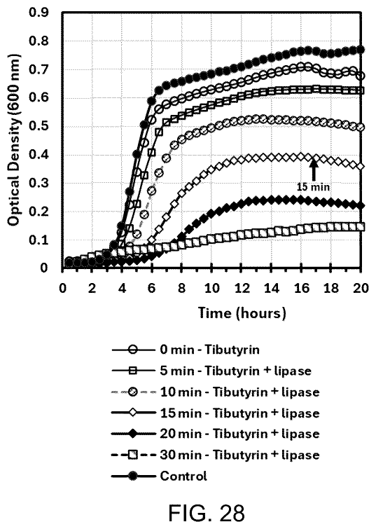

The impact of pancreatic lipase on the hydrolysis of MPG butyrate and tributyrin was investigated and monitored by HPLC over a 120-minute period. The compositions/metabolites of the MPG butyrate and tributyrin after hydrolysis were then characterized. The metabolites/compositions of MPG butyrate after hydrolysis (at 120 minutes) contained mainly monoester and butyric acid, as shown in FIG. 24. In contrast, the products obtained after lipase activity of tributyrin were identified as a mixture of tri-, di-, and monoesters, as well as butyric acid, as shown in FIG. 25. Additionally, the lipase study with tributyrin resulted in diester as the main component in the product after hydrolysis (FIG. 25). The action of pancreatic lipase on the MPG-butyrate and tributyrin clearly affects the growth of the E. coli culture (FIGS. 27 and 28).

Pancreatic lipases are 1,3-regiospecific lipases that cleave specifically at the 1- and 3-positions of triglycerides. By hydrolyzing the ester bonds at the 1- and 3-positions, the enzyme creates two free fatty acids and one molecule of 2-glycerol ester from one triglyceride molecule. The hydrolysis of short-chain fatty acid esters by pancreatic lipase creates short-chain fatty acids, such as butyrate, which are effective at inhibiting bacteria when the acids are protonated (at pH<4). The analysis of the MPG-butyrate lipase reaction reveals that the monoester in MPG-C4 exhibits antimicrobial activity by inhibiting the growth of E. coli by 50% at the neutral pH of the culture (FIG. 26) at time 0. The antimicrobial activity of MPG-butyrate monoester has not been previously reported, making this a novel finding. For the first 20 minutes of the lipase study, the percentage of MPG-butyrate monoester increases, leading to an increase in antimicrobial activity (FIGS. 24 and 26). In contrast, the analysis of the tributyrin lipase reaction shows minimal growth inhibition of 10% (FIG. 26) at the start of the assay (time 0). During the first 45 minutes, the hydrolysis of the tributyrin by lipase mainly results in the production of diester (mostly) and free butyric acid (FIG. 25). The tributyrin (after lipase hydrolysis) showed a slower inhibition effect compared to MPG C4 (FIG. 26).

The antimicrobial data in FIG. 26 show that, after 15 minutes, the MPG-butyrate sample inhibits 92%, while the tributyrin sample inhibits at 53%. The MPG-butyrate antimicrobial activity reaches its maximum at 20 minutes, whereas the tributyrin sample requires a minimum of 45 minutes to achieve its maximum inhibition capacity.

The monoester of MPG-butyrate was found to exhibit antimicrobial activity, and the initial presence of the monoester in MPG-butyrate enables immediate antimicrobial effects. The rapid hydrolysis of the MPG-butyrate diester into monoester is expected to lead to improved antimicrobial efficacy compared to the antimicrobial activity of the tributyrin hydrolysis samples (FIG. 26). The activity of the MPG-butyrate monoester can be beneficial as a feed additive because dietary fat is also a substrate of pancreatic lipase. The fat molecules in the diet will compete for pancreatic lipase, potentially delaying the production of antimicrobial activity from butyrate esters.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

The foregoing descriptions have been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A feed additive comprising an efficacious amount of propylene glycol-butyric acid ester to improve the growth performance or health of a human or animal, wherein the composition possesses a low odor profile compared to butyric acid alone.

2. The feed additive of claim 1, wherein the ester is selected from the group consisting of propylene glycol butyrate ester, propylene glycol valerate ester or propylene glycol ester with one valeric acid and one butyric acid.

3. The feed additive of claim 1, wherein the ester is propylene glycol dibutyrate ester or propylene glycol monobutyrate ester, or mixtures thereof.

4. The feed additive of claim 1, wherein the ester is propylene glycol divalerate ester and/or propylene glycol monovalerate ester, or mixtures thereof.

5. The feed additive of claim 1, wherein the ester is delivered to the animal as fat in the feed.

6. The feed additive of claim 1, wherein the ester is delivered to the animal on a dry carrier.

7. The feed additive of claim 1, wherein the ester is delivered to the animal by spraying the additive directly onto the feed before pelleting.

8. The feed additive of claim 1, wherein the ester is delivered to the animal by spraying the additive directly onto the feed after pelleting.

9. The feed additive of claim 1, further comprising C6-C12 fatty acid MPG-esters.

10. The feed additive of claim 1, wherein the animal is selected from the group consisting of fish, dogs, poultry, pigs, cattle, sheep, goats and horses.

11. A method of improving the growth performance or intestinal health of an animal or human comprising administering to the animal or human an amount of propylene glycol-butyric acid esters effective to improve the growth performance or intestinal health of the animal or human.

12. The method of claim 11, wherein the ester is selected from the group consisting of propylene glycol butyrate ester, propylene glycol valerate ester or propylene glycol ester with one valeric acid and one butyric acid.

13. The method of claim 11, wherein the animal is selected from the group consisting of fish, dogs, poultry, pigs, cattle, sheep, goats and horses.

14. The method of claim 11, wherein the ester is propylene glycol dibutyrate ester or propylene glycol monobutyrate ester, or mixtures thereof.

15. The method of claim 11, wherein the ester is propylene glycol divalerate ester and/or propylene glycol monovalerate ester, or mixtures thereof.

16. A feed additive comprising an efficacious amount of low odor propylene glycol esters that has a reduced odor profile compared to butyric acid alone.

17. A feed additive comprising an efficacious amount of low odor propylene glycol ester that has a lower carbon footprint than commercially available alternatives.

18. A method of providing a sustainable feed additive comprising administering an efficacious amount of low odor propylene glycol ester that is more cost-effective than encapsulated calcium butyrate.

19. The method of claim 18, wherein the ester is selected from the group consisting of propylene glycol butyrate ester, propylene glycol valerate ester or propylene glycol ester with one valeric acid and one butyric acid.

20. The method of claim 18, wherein the animal is selected from the group consisting of fish, dogs, poultry, pigs, cattle, sheep, goats and horses.

21. The method of claim 18, wherein the ester is propylene glycol dibutyrate ester or propylene glycol monobutyrate ester, or mixtures thereof.

22. The method of claim 18, wherein the ester is propylene glycol divalerate ester and/or propylene glycol monovalerate ester, or mixtures thereof.

23. The method of claim 18, wherein the ester is delivered to the animal as fat in the feed.

24. The method of claim 18, wherein the ester is delivered to the animal on a dry carrier.

25. The method of claim 18, wherein the ester is delivered to the animal by spraying the additive directly onto the feed before pelleting.

26. The method of claim 18, wherein the ester is delivered to the animal by spraying the additive directly onto the feed after pelleting.

27. The method of claim 18, further comprising C6-C12 fatty acid MPG-esters.

28. A method of repairing an intestinal barrier in an animal or human comprising administering to the animal or human an amount of propylene glycol-butyric acid esters effective to facilitate repair of the intestinal barrier.

29. A method of reducing mortality due to necrotic enteritis in an animal or human comprising administering to the animal or human an amount of propylene glycol-butyric acid esters effective to reduce the mortality due to necrotic enteritis.

30. A method of reducing an amount of pathogenic bacteria in an animal or human comprising administering to the animal or human an amount of propylene glycol-butyric acid esters effective to reduce the amount of pathogenic bacteria.
